(19) United States
(12) Patent Application Publication
(10) Pub. No.: US 2005/0271650 A1
(43) Pub. Date: Dec. 8, 2005

(54) Method and composition for angiogenesis inhibition

(75) Inventors: Bruce Freimark, Carlsbad, CA (US); Daniel Broek, Henniker, NH (US); Peter Brooks, Carmel, NY (US)

Correspondence Address: WILSON SONSINI GOODRICH & ROSATI, 650 PAGE MILL ROAD, PALO ALTO, CA, 94304-1050, US

(73) Assignee: CellMatrix, Inc.

(21) Appl. No.: 11/061,370

(22) Filed: Feb. 17, 2005

Related U.S. Application Data
(60) Provisional application No. 60/545,376, filed on Feb. 17, 2004.

(51) Int. Cl. A61K 39/395

(52) U.S. Cl. 424/130.1

(57) ABSTRACT

The invention features methods and compositions for inhibiting angiogenesis in a tissue or detecting angiogenesis using antagonists of denatured or proteolyzed forms of laminin.

### RELATED APPLICATIONS

This application claims priority to the U.S. Provisional Application No. 60/545,376, entitled “Method and Composition for Angiogenesis inhibition.”

### FIELD OF THE INVENTION

The invention relates generally to the field of medicine, and relates specifically to methods and compositions for inhibiting angiogenesis in a tissue or detecting angiogenesis using antagonists of denatured or proteolyzed forms of laminin.

### BACKGROUND OF THE INVENTION

Tumor growth and metastasis impacts a large number of people each year. In fact, it was estimated that well over 600,000 new cases of cancer may be diagnosed in a given year in the United States alone (Varner, J. A., Brooks, P. C., and Cheresh, D. A. (1995) Cell Adh. Commun. 3, 367-374). Importantly, numerous studies have suggested that the growth of any solid tumor requires new blood vessel growth for continued expansion of the tumors beyond a minimal size (Varner et al. 1995; Blood, C. H. and Zetter, B. R. (1990) Biochim. Biophys. Acta. 1032:89-118; Weidner, N. et al. (1992) J. Natl. Cancer Inst. 84:1875-1887; Weidner, N. et al. (1991). N. Engl. J. Med. 324:1-7; Brooks, P. C. et al. (1995) J. Clin. Invest. 96:1815-1822; Brooks, P. C. et al. (1994) Cell 79:1157-1164; Brooks, P. C. et al: (1996). Cell 85, 683-693; Brooks, P. C. et al. (1998) Cell 92:391-400. A wide variety of other human diseases also are characterized by unregulated or inappropriate blood vessel development, including ocular diseases such as macular degeneration, cardiovascular disease, psoriasis, and diabetic retinopathy. In addition, numerous inflammatory diseases also are associated with uncontrolled neovascularization, among them are arthritis and psoriasis (Varner et al. 1995).

Angiogenesis is defined as the physiological process by which new blood vessels develop from pre-existing vessels (Varner et al. 1995; Blood and Zetter 1990; Weidner et al. 1992). Angiogenesis is of paramount importance during development, but in the adult it is normally quiescent and triggered only locally and transiently. In adult females, it occurs monthly as part of the reproductive cycle, and in both sexes it is associated with other minor processes such as hair growth and wound healing. This complex process requires cooperation of a variety of molecules including growth factors, cell adhesion receptors, matrix degrading enzymes and extracellular matrix components (Varner et al. 1995; Blood and Zetter 1990; Weidner et al. 1992). Thus, therapies designed to block angiogenesis may significantly effect the growth of solid tumors. In fact, clear evidence has been provided that blocking tumor neovascularization can significantly inhibit tumor growth in various animal models, and human clinical data is beginning to support this contention as well (Varner, J. A., Brooks, P. C., and Cheresh, D. A. (1995) Cell Adh. Commun. 3, 367-374; Folkman, J (2002) Semin. Oncol. 29:15-18). Importantly, numerous studies have suggested that the growth of all solid tumors requires new blood vessel growth for continued expansion of the tumors beyond a minimal size (Varner et al. 1995; Blood and Zetter 1990; Weidner et al. 1992; Weidner et al. 1991; Brooks et al. 1995; Brooks et al. 1994; Brooks et al. 1997; Carmeliet & Jain (2000) Nature Biotech. 407:249-57).

Many investigators have therefore focused their anti-angiogenic approaches towards growth factors and cytokines that initiate and sustain angiogenesis (Varner et al. 1995; Blood and Zetter 1990; Weidner et al. 1992; Weidner et al. 1991; Brooks et al. 1995; Brooks et al. 1994; Brooks et al. 1997). However, there is a large number of distinct growth factors and cytokines with overlapping effects and specificities which have the capacity to stimulate angiogenesis. The therapeutic benefit of blocking any given cytokine may be hampered by this redundancy. Little attention has been directed to other anti-angiogenic targets.

As one might expect, angiogenesis requires proteolytic remodeling of the extracellular matrix (ECM) surrounding blood vessels. There is a suggestion from several studies that this remodeling provides a microenvironment conducive to new blood vessel development (Varner et al. 1995; Blood and Zetter 1990; Weidner et al. 1992; Weidner et al. 1991; Brooks et al. 1995; Brooks et al. 1994; Brooks et al. 1997).

The extracellular protein laminin is a heterotrimeric molecule found primarily, but not exclusively, in the basement membrane (Jones, J. C. R. et al., Micr. Res. Tech. 2000; 51:211-213; Patarroyo, M. et al., Semin. Cancer Biol. 2002; 12:197-207). Each laminin molecule has one copy each of a α-laminin chain, a β-laminin chain, and a γ-laminin chain. There are at least five different alpha chains (molecular weight approximately 400 kDa), three different beta chains (molecular weight approximately 200 kDa), and three different gamma chains of laminin (molecular weight approximately 200 kDa). If they assembled randomly, there would be 45 possible isoforms, but the assembly appears to be biased to favor certain combinations. For example, as far as it is known, the γ2-chain only assembles with the α3- and β3-chains, thereby forming the combination known as laminin-5 (Tunggal et al., Microsc Res Tech. (2000) 51:214-27). As of 2000, 12 isoforms had been characterized. The table below lists the characteristics of these isoforms together with associated GenBank accession numbers.

| | | | | alpha | beta |
|---|---|---|---|---|---|
| Laminin 1 | α1 | β1 | γ1 | P25391 | P07942 |
| Laminin 2 | α2 | β1 | γ1 | | P07942 |
| Laminin 3 | α1 | β2 | γ1 | P25391 | |
| Laminin 4 | α2 | β2 | γ1 | | |
| Laminin 5 | α3 | β3 | γ2 | | |
| Laminin 6 | α3 | β1 | γ1 | | P07942 |
| Laminin 7 | α3 | β2 | γ1 | | |
| Laminin 8 | α4 | β1 | γ1 | | P07942 |
| Laminin 9 | α4 | β2 | γ1 | | |
| Laminin 10 | α5 | β1 | γ1 | O15230 | P07942 |
| Laminin 11 | α5 | β2 | γ1 | O15230 | |
| Laminin 12 | α2 | β1 | γ3 | P24043 | P07942 |

Based on structural studies, generally the carboxy-terminal regions of the beta and gamma chains associate a portion of the alpha chain in a long rod-like structure. The amino-terminal domains project from the trimeric structure as monomeric arms, and the carboxy-terminal region of the continues out from the trimeric structure as a globular G domain.

As mentioned above, it has been proposed that inhibition of angiogenesis would be a useful therapy for restricting tumor growth, especially as an alternate means for tackling multi-drug resistant tumors. Also, since the intended target for the angiogenesis-inhibitory agents, endothelial cells, are not cancerous, they are unlikely to mutate to acquire drug resistance. Further, anti-angiogenesis drugs should have minimal side effects as angiogenesis is already down-regulated in adults.

Means for inhibition of angiogenesis that have been proposed by others include: (1) inhibition of release of “angiogenic molecules” such as βFGF (fibroblast growth factor), (2) neutralization of angiogenic molecules, such as by use of anti-βFGF antibodies, and (3) inhibition of endothelial cell response to angiogenic stimuli. This latter strategy has received significant attention, and Folkman and co-workers (Cancer Biology, 3:89-96 (1992)) have described several endothelial cell response inhibitors, including collagenase inhibitors, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like that might be used to inhibit angiogenesis. (For additional proposed inhibitors of angiogenesis, see Blood and Zetter 1990; Moses et al. (1990) Science 248:1408-1410; Ingber et al. (1988) Lab. Invest., 59:44-51; and U.S. Pat. Nos. 5,092,885, 5,112,946, 5,192,744, and 5,202,352.)

Further, some of the factors identified that modulate angiogenesis are fragments of larger proteins that have other functions. Thus, angiostatin is a 38 kDa fragment of plasminogen and endostatin is a 20 kDa fragment of collagen. Additionally, another protein, PEX, appears to retard angiogenesis. Paradoxically, PEX is a fragment of MMP-2, a metalloprotease that is strongly implicated in allowing and promoting angiogenesis by degrading the ECM. Similarly, another inhibitor of angiogenesis and metastasis, Tn-1, is fragment of troponin.

### SUMMARY OF THE INVENTION

The present invention provides antagonists of denatured or proteolyzed laminins that can inhibit angiogenesis. Antagonists of the present invention specifically bind to a denatured or proteolyzed laminin, but bind with substantially reduced affinity to native forms of the same laminin. Antagonists can be specific for any form or denatured laminin, including denatured laminin. Antagonists can be specific for any of the five different alpha chains, any of the three different beta chains, any of the three different gamma chains of laminin, or any laminin chains subsequently discovered.

An antagonist may be an antibody, or functional fragment thereof, that binds via the variable region with denatured laminin but binds via the variable region to a substantially lesser extent with the native form of the laminin. Such antibodies can be monoclonal or polyclonal. An antagonist also can be a polypeptide or peptide with specificity for a denatured laminin, but less affinity for a native form of the laminin. Antagonists also can be non-peptidic compounds such as small organic molecules, carbohydrates, or oligonucleotides.

The invention therefore describes methods for inhibiting angiogenesis in a tissue comprising administering to the tissue a composition comprising an angiogenesis-inhibiting amount of an antagonist of the invention. The antagonist of the invention may be administered by itself, in a composition, or with another therapeutic compound.

The tissue to be treated can be any tissue in which inhibition of angiogenesis is desirable, such as diseased tissue, where neo-vascularization is occurring. Exemplary tissues include inflamed tissue, solid tumors, metastases, tissues undergoing fibrosis, tissues undergoing vasculitis, hemangiomas, tissues undergoing restenosis, and the like.

The invention also provides methods for detecting angiogenesis in a tissue by contacting an antagonist of the invention with the tissue or contacting an antagonist of the invention with blood or serum derived from the organism comprising the tissue in question. Such methods are appropriate for use both ex vivo and in vivo.

Methods also are provided for detecting tumorous tissue, metastases, and tumor invasion into a tissue by contacting an antagonist of the invention with a tissue either ex vivo or in vivo, or contacting an antagonist of the invention with blood or serum derived from the organism comprising the tissue in question.

The invention also provides methods for screening antagonists that bind specifically to a denatured laminin or laminins, but bind with substantially reduced affinity to the native form of the laminin or laminins. Such antagonists may be used to inhibit angiogenesis.

In one aspect, the present invention features an antagonist that specifically binds to a denatured laminin or laminins but binds to the native form of each of the aforementioned laminin or laminins with substantially reduced affinity. In some embodiments, the antagonist has at least about a two-fold increased binding to denatured laminin when compared to its binding to native laminin. In another embodiment, the antagonist has at least about a five-fold increase in binding between denatured laminin compared to its binding to native laminin. In still another embodiment, the antagonist has at least about a six-fold increase in binding between denatured laminin compared to its binding to native laminin. In a further embodiment, the antagonist has at least about a ten-fold increase in binding between denatured laminin compared to its binding to native laminin.

In another aspect, the present invention features an antagonist that specifically binds to a backbone domain in denatured laminin but binds the aforementioned backbone domain in the native form of said laminin with an affinity that is less by a factor of at least about two. In an embodiment, the antagonist that specifically binds to a backbone domain in denatured laminin but binds the aforementioned backbone domain in the native form of said laminin with an affinity that is less by a factor of at least about five. In another embodiment, the antagonist that specifically binds to a backbone domain in denatured laminin but binds the aforementioned backbone domain in the native form of said laminin with an affinity that is less by a factor of at least about six. In a further embodiment, the antagonist that specifically binds to a backbone domain in denatured laminin but binds the aforementioned backbone domain in the native form of said laminin with an affinity that is less by a factor of at least about ten.

In a preferred embodiment of aspects of the invention, the antagonist is a monoclonal antibody. In still further preferred embodiments, the antagonist is a monoclonal antibody having the binding specificity of monoclonal antibody clone LMD2, LMD9, LMD21, LMD24, LMD52, LMD105, LMD1, LMD5, LMD11, LMD17, LDM26, LMD13, LMD14, LMD15, LMD16, LMD23, LMD30, LMD209, or LMD418. In other embodiments, the antagonist of the invention is a polyclonal antibody. In further embodiments, the antagonist of the invention is a humanized monoclonal antibody. In still further embodiments, the antagonist of the invention is a chemically modified monoclonal antibody. In yet other embodiments, the antagonist of the invention is a fragment of a monoclonal antibody.

In other preferred embodiments of aspects of the invention, the antagonist of the invention is a polypeptide, a linear peptide, or a cyclic peptide. In other embodiments of aspects of the invention, the antagonist of the invention is a non-peptidic compound. In further embodiments of aspects of the invention, the antagonist of the invention is an oligonucleotide, a carbohydrate, a lipid, or a synthetic polymer.

In some embodiments on the invention, the aforementioned antagonist can be conjugated to a cytotoxic agent, a cytostatic agent, or a radioisotope.

In another aspect, the present invention features a method of inhibiting angiogenesis in a tissue comprising the step of administering to the aforementioned tissue an antagonist of the invention. The antagonist may be administered intravenously, transdermally, intrasynovially, intramuscularly, intratummorally, intraoculory, intranasally, intraarticularly, intrathecally, topically, or orally. The antagonist may be further administered in conjunction with chemotherapy or radiation therapy. In some embodiments, the antagonist of the invention is conjugated with a radioisotope.

In some embodiments, the tissue to be treated is inflamed and angiogenesis is occurring. In other embodiments, the tissue is present in a mammal. In further embodiments, the tissue is arthritic tissue, ocular tissue, retinal tissue, a hemangioma, lung tissue, kidney tissue, or vascular tissue.

In a further aspect of this invention, the invention features a method of inhibiting tumor growth, tumor metastasis, or metastasized tumor growth in a tissue that includes the step of administering the antagonist of the invention; this method may include additional steps. In this aspect, the antagonist of the invention may be administered intravenously, transdermally, intrasynovially, intramuscularly, intratummorally, intraoculory, intranasally, intraarticularly, intrathecally, topically, or orally. In certain embodiments, this antagonist of the invention is administered in conjunction with chemotherapy, is administered in conjunction with radiation therapy, or is administered after the antagonist is first conjugated to a radioisotope.

In certain embodiments, this tumor growth, tumor metastasis, or metastasized tumor growth is a melanoma, a carcinoma, a sarcoma, a fibrosarcoma, a glioma, or an astrocytoma. In some embodiments of this invention, this method of treatment comprises the step of heating the tissue.

In a further aspect of this invention, the invention features a method of inhibiting psoriasis, macular degeneration, restenosis, rheumatoid arthritis, scleraderma, fibrosis, or vasculitis in a tissue that includes the step of administering the antagonist of the invention; this method may include additional steps. In this aspect, the antagonist of the invention may be administered intravenously, transdermally, intrasynovially, intramuscularly, intratummorally, intraoculory, intranasally, intraarticularly, intrathecally, topically, or orally. In certain embodiments, this antagonist of the invention is administered in conjunction with chemotherapy, is administered in conjunction with radiation therapy, or is administered after the antagonist is first conjugated to a radioisotope. In some embodiments of this invention, this method of treatment comprises the step of heating the tissue.

In an additional aspect to this invention, the invention features a method of detecting angiogenesis in an organism by contacting a tissue, blood, or serum from said organism with an antagonist of this invention; this method of detecting angiogenesis may include other steps. In some embodiments, the contacted tissue, blood, or serum is ex vivo. In other embodiments, the contacted tissued, blood, or serum is in vivo. In further embodiments, the contacted tissued, blood, or serum is in vivo and the antagonist of the invention is administered intravenously, transdermally, intrasynovially, intramuscularly, intratummorally, intraoculory, intranasally, intraarticularly, intrathecally, topically, or orally. In certain embodiments of this aspect, or other aspects, the antagonist is conjugated to a fluorochrome, a radioactive tag, a paramagnetic heavy metal, a diagnostic dye, a quantum dot, or an enzyme.

In a still further aspect to this invention, the invention features a method of detecting a tumor, a tumor invasion, or metastasis in an organism by contacting a tissue, blood, or serum from said organism with an antagonist of this invention; this method of detection may include additional steps. In some embodiments, the contacted tissued, blood, or serum is ex vivo. In other embodiments, the contacted tissued, blood, or serum is in vivo. In further embodiments, the contacted tissued, blood, or serum is in vivo and the antagonist of the invention is administered intravenously, transdermally, intrasynovially, intramuscularly, intratummorally, intraoculory, intranasally, intraarticularly, intrathecally, topically, or orally. In certain embodiments of this aspect, or other aspects, the antagonist is conjugated to a fluorochrome, a radioactive tag, a paramagnetic heavy metal, a diagnostic dye, a quantum dot, or an enzyme.

In an additional aspect, the invention features a method of screening for denatured laminin antagonists comprising the steps of: (a) providing a candidate antagonist; (b) denaturing the laminin of interest by a thermal, chemical, or enzymatic denaturation means; (c) measuring the candidate antagonist's first affinity for a denatured laminin molecule; (d) measuring the candidate antagonist's second affinity for a native laminin molecule; (e) selecting the candidate antagonist as a denatured laminin antagonist if the first affinity binding affinity with the denatured laminin is greater than the second affinity with the native laminin. In one embodiment, the candidate antagonist is a small organic compound. In another overlapping embodiment, the candidate antagonist is a non-peptidic compound, an oligonucleotide, a polypeptide, a linear polypeptide, or a cyclic peptide, a monoclonal antibody, or a polyclonal antibody. In other embodiments of this aspect, and other aspects, the affinities of binding are measured by a radioimmunoassay, FRET, or an enzyme-linked immunosorbent assay (ELISA).

In some embodiments, a candidate antagonist specifically binds to denatured laminin with an affinity that is greater by a factor of at least about two than the affinity of binding to the native form of the laminin. In other embodiments, a candidate antagonist specifically binds to denatured laminin with an affinity that is greater by a factor of at least about five than the affinity of binding to the native form of the laminin. In further embodiments, a candidate antagonist specifically binds to denatured laminin with an affinity that is greater by a factor of at least about six than the affinity of binding to the native form of the laminin. In still other embodiments, a candidate antagonist specifically binds to denatured laminin with an affinity that is greater by a factor of at least about ten than the affinity of binding to the native form of the laminin.

In another aspect, the invention features a method of detecting denatured laminin in the blood using an antagonist of the invention. In certain embodiments of this invention, the method of detecting denatured laminin in the blood is used to detect tissue damage.

In a further aspect, the invention features a method of screening for modulators of tissue damage wherein a test compound is administered to an organism and the amount of circulating denatured laminin is measured by the means of binding to an antagonist of the invention.

In an additional aspect, the invention features a method of screening for modulators of inflammation wherein a test compound is administered to an organism and the amount of circulating denatured laminin is measured by the means of binding to the antagonist of the invention.

In yet another aspect, the invention features a method of screening for modulators of angiogenesis wherein a test compound is administered to an organism and the amount of circulating denatured laminin is measured by the means of binding to the antagonist of the invention.

In still another aspect, the invention features a method of inhibiting cell adhesion in a tissue comprising administering the antagonist of the invention.

In another aspect, the invention features a method of inhibiting cell migration comprising administering the antagonist of the invention.

In yet another aspect, the instant invention features a method of treating or preventing an abnormal condition comprising the step of administering an antagonist of the invention. In an embodiment of this aspect, as well as others, the said abnormal condition is selected from the group consisting of autoimmune diseases and inflammation. In another embodiment of this aspect, as well as others, an antagonist of the invention is administered together with a secondary monoclonal antibody.

In yet another aspect, the invention features a method for screening for denatured laminin antagonists comprising selecting an antagonist by the ability of the candidate antagonist to compete with the antibody from the LMD2 clone for binding to denatured laminin. In other embodiments, monoclonal antibodies that exhibit preferential binding for denatured laminin with respect to native laminin may be used in this method.

The antagonist of the invention may bind to the backbone domain, or the polypeptide chain, in the denatured laminin with an affinity that is two-fold, five-fold, six-fold, or ten-fold greater than its binding to the backbone domain of the native laminin.

In an additional aspect, the invention features an epitope of laminin wherein the epitope is bound by an antibody or antagonist of the invention with a greater affinity in denatured laminin than the antibody or antagonist binds native the epitope in native laminin. The epitope may be a purified or isolated peptide. The epitope may comprise amino acids other than those of the epitope. The epitope may also consist essentially of the amino acids of the epitope. If the epitope comprises amino acids in addition to those of the epitope, it may comprise about 1 to 10 additional amino acids, it may comprise about 10 to 33 additional amino acids, it may comprise about 30 to 50 additional amino acids, it may comprise about 40 to 66 additional amino acids, or it may comprise about 50 to 100 additional amino acids, or it may comprise more than about 100 additional amino acids.

In all of the aspects of the invention, the methods described herein may also be used in the preparation of a medicament for treating the conditions described herein.

### DETAILED DESCRIPTION OF THE INVENTION

Angiogenesis inhibitors hold great promise as therapies for solid tumors. Degraded extracellular matrix (ECM) proteins play an active and essential role in angiogenesis and cryptic epitopes that are only exposed upon proteolytic remodeling play a critical and essential role. Denaturation of the native three dimensional structure of mature laminin may expose cryptic regulatory regions that control or modulate angiogenesis. Cryptic sites in laminin therefore are expected to play an active role in angiogenesis, probably by engaging endothelial cell surface receptors and thereby modifying the behavior of the endothelial cells. Modulation of or binding to these cryptic regulatory regions will provide heretofore unrecognized means for the diagnosis and/or the inhibition of angiogenesis. Antagonists of these laminin cryptic sites will block angiogenesis/tumor growth as remodeling of the ECM is necessarily extensive at such sites. Monoclonal antibodies to degraded ECM proteins, such as laminin, may also prove useful for target drug delivery. Further, such monoclonal antibodies could have a diagnostic use for detecting the degraded ECM protein fragments in sera of patients with angiogenic conditions.

Angiogenesis is the development of a new vessel from the existing vasculature. In this process, endothelial cells lining vessels are activated by cytokines (Klagsbrun, M. (1992) Seminars in Cancer Biology 3 (2), 81-7; Neufeld, G., Cohen, T., Gengrinovitch, S., and Poltorak, Z. (1999) FASEB Journal 12 (1), 9-22). The activated endothelial cells proliferate and secrete proteases that degrade the basement membrane surrounding the vessel. During the early stages of angiogenesis, activated endothelial cells are thought to invade through proteolytically modified subendothelial basement membrane (Kleiner, D. E., and Stetler-Stevenson, W. G. (1999) Cancer Chemotherapy & Pharmacology 43 Suppl., S42-51; Birkedal-Hansen, H. (1995) Current Opinion in Cell Biology 7(5), 728-35). Interstitial collagens are also degraded as the endothelial cells migrate into the interstial space. During the maturation phase of angiogenesis, activated endothelial cells undergo morphogenesis and reorganize into capillary-like tubes. Vascular smooth muscle cells are recruited to form a sheath around the tube. Eventually the endothelial cells secrete new ECM components, form associations with accessory cells such as pericytes, and differentiate into mature vessels (Hirschi, K. K., Rohovsky, S. A., and D'Amore, P. A. (1997) Transplant Immunology 5(3), 177-8; Lampugnani, M. G., and Dejana, E. (1997) Current Opinion in Cell Biology 9(5), 674-82).

Angiogenesis depends on the coordinated activity of a number of distinct families of molecules. Each of these classes represents a set of potential targets for antiangiogenic therapies. Some of the well-studied angiogenic factors include growth factors and their receptors, proteolytic enzymes; cell adhesion molecules, and ECM components (Leek, R. D., Harris, A. L., and Lewis, C. E. (1994) Journal of Leukocyte Biology 56(4), 423-35; Mignatti, P., and Rifkin, D. B. (1996) Enzyme & Protein 49(1-3), 117-37; Pepper, M. S., and Montesano, R. (1990) Cell Differentiation & Development 32(3), 319-27; Brooks, P. C. (1996) European Journal of Cancer 32A (14), 2423-9; Brooks, P. C. (1996) Cancer & Metastasis Reviews 15(2), 187-94; Luscinskas, F. W., and Lawler, J. (1994) FASEB Journal 8(12), 929-38; Carey, D. J. (1991) Annual Review of Physiology 53, 161-77; Ingber, D. E., and Folkman, J. (1989) Cell 58(5), 803-5; O'Reilly, M. S., Holmgren, L., Shing, Y., Chen, C., Rosenthal, R. A., Moses, M., Lane, W. S., Cao, Y., Sage, E. H., and Folkman, J. (1994) Cell 79(2), 315-28). Importantly, these families of molecules do not function in isolation but are rather connected in complex networks that function cooperatively to regular new blood vessel development. Blocking the activities of essential members of these four classes of angiogenesis regulators has been shown to block angiogenesis.

Matrix-Remodeling Proteases: During the invasive stage of angiogenesis, the activated endothelial cells utilize a number of matrix altering enzymes to remodel the local ECM (Kleiner & Stetler-Stevenson (1999); Birkedal-Hansen, H. (1995)). This proteolytic remodeling helps to create a microenvironment that is conducive to new blood vessel development. Matrix metalloproteases (MMPs) are thought to play a critical role in remodeling of basement membranes and the interstitial ECM. In addition, serine proteases have also been shown to play a critical role in angiogenesis. Inhibitors of these MMPs are currently being explored as potential antiangiogenic therapies. Because the MMPs likely play important roles at non-angiogenic sites, MMP-inhibitors may have deleterious side effects.

Integrins: The connection between the ECM (native and degraded) and vascular cells is predominately mediated by a class of cell surface receptors termed integrins (Brooks, P. C. (1996) European Journal of Cancer; Brooks, P. C. (1996) Cancer & Metastasis Reviews; Luscinskas & Lawler (1994)). Integrins are a family of cell surface heterodimers composed of a and β chains that mediate cellular interactions with both ECM components and other cells (Id.).

Studies have provided evidence that integrins play an important role in the regulation of vascular cell adhesion and migration (Id.). Whereas, the native ECM interacts with the vascular cells through one set of integrins, the degraded ECM interacts with a distinct (but overlapping) set of integrins. The alteration of integrin interactions upon protoelytic remodeling of the ECM is likely to result in altered cellular signaling and promote and processes of proliferation and migration of the vascular cells.

Degraded Matrix Proteins: The MMP-mediated degradation of the ECM proteins exposes sites which, in the native proteins, are either buried in the core of the native protein or which have distinct structures. It is now apparent that the exposure of some cryptic sites plays a critical role in the process of angiogenesis. Antagonists of these cryptic sites block essential signals that are required for angiogenesis.

The ECM is very stable; for example, the half-life of many collagens is longer than one year. Consequently, while degraded ECM proteins are found in abundance at angiogenic sites, they are rarely found in normal tissues. For example, a monoclonal antibody (Mab HUIV26), which reacts specifically with denatured Type IV collagen but not native forms of collagen, stains angiogenic sites in tumor tissue but fails to stain normal tissue. The extensive remodeling of the ECM at angiogenic sites and lack thereof in normal tissues has important implications for diagnostic and therapeutic application of antagonists of the degraded ECM. First, the degraded ECM proteins produced during angiogenesis are likely to find their way into the blood stream. Monoclonal antibodies that specifically recognize these degraded ECM proteins may therefore prove useful as diagnostic tool for diseases with enhanced angiogenesis, by monitoring serum for the presence of a specific angiogenic marker. Second, because the degraded ECM proteins appear in high concentration at angiogenic sites in tumors, but at very low levels in surrounding normal tissue, degraded-ECM-specific monoclonal antibodies could be used as vehicles for delivery of toxic or imaging molecules to tumor sites. By conjugating cytotoxins to such monoclonal antibodies, the cytotoxins could be delivered to the tumor. By conjugating imaging probes (paramagnetic molecules with MRI applications), the monoclonal antibodies could be used in noninvasive detection of angiogenic sites. Third, systemic administration of Mab HUIV26, but not a control isotype matched antibody, inhibits bFGF induced angiogenesis in chick CAM assays (<90% inhibition), and inhibits tumor growth in a variety of mouse tumor models (50-90% inhibition). These results demonstrate that the degradation/denaturation of Type IV collagen exposes sites which play an active and essential role in angiogenesis. Thus, antagonists of other degraded ECM proteins, such as laminin, may prove useful in the treatment of diseases where aberrant angiogenesis is a critical factor in the disease.

For an example of the use of laminin chain-specific probes for the detection of invasive cells, such as cancer cells, see U.S. Pat. Nos. 5,660,982 and 6,143,505 and U.S. Publication No. 2005/0019332 A1, by Tryggvason et al.

Degradation/denaturation of other ECM molecules also exposes critical sites that play a necessary role in angiogenesis. For example, Mab XL313 that recognizes denatured Type I collagen, but not native Type I collagen, is a potent inhibitor of angiogenesis in CAM assays, and blocks tumor growth in mouse tumor models. However, it is unlikely that all monoclonal antibodies that recognize denatured Type I or IV collagen block angiogenesis. Further, it is likely that the degradation of additional ECM molecules, other than collagen, results in exposure of sites that play an essential role in angiogenesis and tumor growth. Recent studies suggest that distinct domains present within ECM proteins regulate angiogenesis and tumor growth. In fact, fragments of molecules such as plasminogen (i.e. angiostatin), collagen-XVIII (i.e. endostatin), collagen-XV (i.e. Restin), MMP-2 (i.e. PEX-domain), fibronectin, and thrombospondin have all been shown to regulate angiogenesis (O'Reilly, M. S., Holmgren, L., Shing, Y., Chen, C., Rosenthal, R. A., Moses, M., Lane, W. S., Cao, Y., Sage, E. H., and Folkman, J. (1994) Cell 79(2), 315-28; O'Reilly, M. S., Boehm, T., Shing, Y., Fukai, N., Vasios, G., Lane, W. S., Flynn, E., Birkhead, J. R., Olsen, B. R., and Folkman, J. (1997) Cell 88(2), 277-85; Ramchandran, R., Dhanabal, M., Volk, R., Waterman, M. J., Segal, M., Lu, H., Knebelmann, B., and Sukhatme, V. P. (1999) Biochemical & Biophysical Research Communications 255(3), 735-9; Brooks, P. C., Silletti, S., von Schalscha, T. L., Friedlander, M., and Cheresh, D. A. (1998) Cell 92(3), 391-400; Castellani, P., Viale, G., Dorcaratto, A., Nicolo, G., Kaczmarek, J., Querze, G., and Zardi, L. (1994) International Journal of Cancer 59(5), 612-8; Tolsma, S. S., Volpert, O. V., Good, D. J., Frazier, W. A., Polyerini, PlJ., and Bouck, N. (1993) Journal of Cell Biology 122(2), 497-511).

Evidence for an Active Role of the ECM in Angiogenesis

The ECM, in a vastly simplified fashion, can be characterized as being composed of two general compartments. Embracing this two-compartment concept, the ECM can be divided into the interstitial ECM and the basal lamina or casement membrane. The basement membrane is a specialized form of ECM that separates both epithelia and endothelia from their underlying mesenchyme (Timpl, R. (1989) European Journal of Biochemestry 180(3), 487-502; Timpl, R., and Brown, J. C. (1996) Bioessays 18(2), 123-32; Yurchenco, P. D., and Schittny, J. C. (1990) FASEB Journal 4(6), 1577-90; Schittny, J. C., and Yurchenco, P. D. (1989) Current Opinion in Cell Biology 1(5), 983-8). The major components of the basement membrane include laminin, Type IV collagen, enactin/nidogen, SPARC, perlecan, as well as other proteoglycans. These components exhibit a complex pattern of molecular interconnections and supramolecular assemblies that are organized into a mesh-like network (Id.).

The mesh-like network of the basement membrane is connected to the underlying interstitial matrix by a series of anchoring fibers including collagen-VII and fibrilin (Id.). Some of the well-characterized components include a variety of genetically distinct forms of collagen, such as collagen-I, II, III, and V. In addition, a number of non-collagenous glycoproteins also help compose the interstitial matrix including fibronectin, gibrinogen/fibrin, thrombospondin, and vitronectin (Adechi, E., Hopkinson, I., and Hayashi, T. (1997) International Review of Cytology 173, 73-156; Mosher, D. F., Sottile, J., Wu, C., and McDonald, J. A. (1992) Curr. Opion. Cell Biol. 4, 810-818). Finally, a number of proteoglycans also contribute to the complex architecture of the interstitial matrix. The networks of proteins that make up the ECM in conjunction with integrins function cooperatively to regulate new blood vessel development.

Historically, the ECM was thought to provide mechanical and structural support to cells and tissues. However, following the development of new molecular, cellular and biochemical techniques, this limited view of the ECM has expanded dramatically. In fact, the ECM can be defined in broad terms as a complex interconnected network of fibrous proteins, proteoglycans and structural glycoproteins that provide both mechanical and biochemical regulatory functions to cells and tissues. In angiogenesis, the regulatory information contained within the three dimensional structure of the ECM must be recognized and transferred to recipient cells capable of forming new blood vessels. To this end, integrin-mediated ligation of ECM components has been shown to activate distinct signal transduction pathways, which in turn may regulate neovascularization. As discussed above, several monoclonal antibodies that specifically recognize the denatured form of collagen are potent inhibitors of angiogenesis. Below is a summary of the key observations about one of these monoclonal antibodies which illustrates the importance of degraded ECM proteins in the regulation of angiogenesis and tumor growth.

A highly specific monoclonal antibody termed HUIV26 that reacts with denatured collagen-IV, but not native triple helical collagen-IV, was developed by the method of subtractive immunization as set forth in this specification. This antibody, Mab HUIV26, specifically binds to denatured human collagen-IV but shows little if any reactivity to the native protein. Additionally, this monoclonal antibody fails to interact with other ECM components including fibronectin, vitronectin, fibrinogen, native or denatured collagen-I.

Two other monoclonal antibodies have been characterized that block angiogenesis that specifically recognize cryptic sites in other ECM proteins. This suggests that the native ECM contains a wealth of cryptic sites that play important roles in supporting angiogenesis.

Proteases such as MMPs and serine proteases appear to play a critical role in exposing cryptic sites. Fragments of molecules such as plasminogen, collagen-XVIII, collagen-XV, MMP-2, fibronectin, and thrombospondin have all been shown to regulate angiogenesis (O'Reilly, M. S., Holmgren, L., Shing, Y., Chen, C., Rosenthal, R. A., Moses, M., Lane, W. S., Cao, Y., Sage, E. H., and Folkman, J. (1994) Cell 79(2), 315-28; O'Reilly, M. S., Boehm, T., Shing, Y., Fukai, N., Vasios, G., Lane, W. S., Flynn, E., Birkhead, J. R., Olsen, B. R., and Folkman, J. (1997) Cell 88(2), 277-85; Ramchandran, R., Dhanabal, M., Volk, R., Waterman, M. J., Segal, M., Lu, H., Knebelmann, B., and Sukhatme, V. P. (1999) Biochemical & Biophysical Research Communications 255(3), 735-9; Brooks, P. C., Silletti, S., von Schalscha, T. L., Friedlander, M., and Cheresh, D. A. (1998) Cell 92(3), 391-400; Castellani, P., Viale, G., Dorcaratto, A., Nicolo, G., Kaczmarek, J., Querze, G., and Zardi, L. (1994) International Journal of Cancer 59(5), 612-8; Tolsma, S. S., Volpert, O. V., Good, D. J., Frazier, W. A., Polyerini, PlJ., and Bouck, N. (1993) Journal of Cell Biology 122(2), 497-511). These fragments may serve as antagonists of cell surface receptors regulating angiogenesis.

In the instant invention, angiogenesis can be inhibited by antagonizing certain cryptic sites found in the ECM component laminin. In the instant invention, monoclonal antibodies. or other antagonists, that react specifically with cryptic sites in laminin are identified and their antiangiogenic activity is tested.

Laminin itself is a large heterotrimer of approximately 900 kDa composed of three distinct chains termed α; β and γ (Yurchenco, P. D., and Ruben, G. C. (1987) Journal of Cell Biology 105(6 Pt 1), 2559-68; Yurchenco, P. D., Cheng, Y. S., and Colognato, H. (1992) Journal of Cell Biology 117(5), 1119-33; Engvall, E. (1993) Kidney International 43(1), 2-6; Ekblom, M., Falk, M., Salmivirta, K., Durbeej, M., and Ekblom, P. (1998) Annals of the New York Academy of Sciences 857, 194-211; Mayer, U., Kohfeldt, E., and Timpl, R. (1998) Annals of the New York Academy of Sciences 857, 130-42). Laminin has been shown to support cell adhesion, migration, regulate signal transduction, gene expression and differentiation (Id.). Laminin is known to support endothelial cell interactions by both integrins and non-integrin receptors (Schnaper, H. W., Kleinman, H. K., and Grant, D. S. (1993) Kidney International 43(1), 20-5; Basson, C. T., Knowles, W. J., Bell, L., Albelda, S. M., Castronovo, V., Liotta, L. A., and Madri, J. A. (1990) Journal of Cell Biology 110(3), 789-801; Nomizu, M., Kuratomi, Y., Malinda, K. M., Song, S. Y., Miyoshi, K., Otaka, A., Powell, S. K., Hoffman, M. P., Kleinman, H. K., and Yamada, Y. (1998) Journal of Biological Chemistry 273(49), 32491-9). Previous studies by Grant and others demonstrated that different functional domains within laminim regulate distinct endothelial cell processes (Grant, D. S., Tashiro, K., Segui-Real, B., Yamada, Y., Martin, G. R., and Kleinman, H. K. (1989) Cell 58(5), 933-43). In fact, it was shown that an RGD tripeptide-containing domain within the α chain of laminim promotes endothelial cell adhesion (Id.). This adhesive-promoting ability was shown to be dependent on ligation of β1 integrins. Moreover, a second domain within the β1 chain of laminin (YIGSR) was shown to induce cell-cell interactions, regulate morphogenesis, and induce reorganization of endothelial cells into tube-like structures (Grant, D. S., Tashiro, K., Segui-Real, B., Yamada, Y., Martin, G. R., and Kleinman, H. K. (1989) Cell 58(5), 933-43; Kubota, Y., Kleinman, H. K., Martin, G. R., and Lawley, T. J. (1988) Journal of Cell Biology 107(4), 1589-98). When added in soluble form, this peptide significantly inhibited angiogenesis (Id.). Interestingly, endothelial cells have been shown to bind to this YIGSR sequence via interactions with the non-integrin laminin receptor (Ruoslahti, E., and Engvall, E. (1997) Journal of Clinical Investigation 100 (11 Suppl), S53-6). In further studies, a laminin-derived peptide from the al chain (IKVAV) was shown to regulate protease activity and promote angiogenesis (Kibbey, M. C., Grant, D. S., and Kleinman, H. K. (1992) Journal of National Cancer Institute 84(21), 1633-8; Grant, D. S., Kinsella, J. L., Fridman, R., Auerbach, R., Piasecki, B. A., Yamada, Y., Zain, M., and Kleinman, H. K. (1992) Journal of Cellular Physiology 153(3), 614-25). Finally, other laminin peptides were also shown to promote endothelial cell adhesion and migration in vitro (Malinda, K. M., Nomizu, M., Chung, M., Delgado, M., Kuratomi, Y., Yamada, Y., Kleinman, H. K., and Ponce, M. L. (1999) FASEB Journal 13(1), 53-62; Ponce, M. L., Nomizu, M., Delgado, M. C., Kuratomi, Y., Hoffman, M. P., Powell, S., Yamada, Y., Kleinman, H. K., and Malinda, K. M. (1999) Circulation Research 84(6), 688-94). Taken together, several distinct sequences within laminin may function to regulate angiogenesis both positively and negatively.

While many of the angiogenesis-regulating sequences within native laminin are accessible to cells, it is also possible that biologically relevant sequences or domains may be cryptic, and require proteolytic remodeling for exposure. To this end, recent studies by Giannelli and others demonstrated that MMP-2 mediated cleavage of laminin-5, exposed a cryptic site within the γ2 subunit, which in turn induced cellular motility (Giannelli, G., Falk-Marzillier, J., Schiraldi, O., Stetler-Stevenson, W. G., and Quaranta, V. (1997) Science 277(5323), 225-8; Giannelli, G., Pozzi, A., Stetler-Stevenson, W. G., Gardner, H. A., and Quaranta, V. (1999) American Journal of Pathology 154(4), 1193-201). Thus, it is possible that cryptic sites within the three dimensional structure of laminin may regulate angiogenesis as well.

Degraded fragments of the ECM play an active role in angiogenesis. Monoclonal antibodies that react specifically to degrade ECM molecules and which block angiogenesis have been identified. Importantly, some of these antibodies appears to block unique steps in the process of angiogenesis. Evidence for this comes from the observation that the characterized monoclonal antibodies behave differently with respect to their effects on endothelial cells. For example, while two antibodies to denatured collagen both block angiogenesis, only one of them is a potent inhibitor of endothelial cell adhesion. Thus, while the latter epitope may be important for adhesion, the other epitope appears to be required for a different event in angiogenesis. This suggests that the degradation of the ECM during the early phases of angiogenesis exposes a wealth of cryptic sites that play unique functional role in promoting angiogenesis. It is possible that tumor growth can be most effectively blocked by antagonizing several of these distinct cryptic sites in laminin. Monoclonal antibodies and other antagonists are identified that recognize the degraded/denatured forms of other ECM proteins. These will be useful in:
1. Inhibiting angiogenesis;
2. Inhibiting tumor growth;
3. Targeting delivery of cytotoxins to (angiogenic) tumor sites;
4. Treatment of ocular vascular diseases;
5. Treatment of other diseases with abnormal vessel development (e.g. rheumatic arthritis);
6. Detection of angiogenic sites in situ;
7. Detection of angiogenic sites in vivo (via conjugates with imaging molecules);
8. Detection of angiogenic markers in serum or urine; and
9. Basic science research in understanding the role of the ECM in angiogenesis.

The present invention features antagonists of denatured or proteolyzed laminins that inhibit angiogenesis. Antagonists of the present invention specifically bind to denatured or proteolyzed laminin molecules, but bind with substantially reduced affinity to the native form. Antagonists of the invention may be specific for any denatured laminin, including denatured alpha-laminin or beta-laminin. Antagonists can be specific for any of the five different alpha chains, any of the three different beta chains, any of the three different gamma chains of laminin, or any laminin chains subsequently discovered.

An antagonist may be an antibody, or functional fragment thereof, that binds via its variable region with denatured laminin but binds to a substantially lesser extent with the native form of the laminin. The antibodies of the invention can be monoclonal or polyclonal; means for isolating such antibodies are described below. An antagonist of the invention can also be a non-antibody molecule capable of specifically binding denatured laminin, but binding a native form of the laminin with less affinity. Such non-antibody antagonists also can be other proteins, other polypeptides, or non-peptidic compounds such as a small organic molecules, carbohydrates, or oligonucleotides.

For an example of such a peptide antagonist, see U.S. Publication No. 20040224896 A1 by Brooks and Akalu entitled “STQ Peptides.”

The invention also provides methods for screening antagonists that bind specifically to a denatured laminin or laminins, but bind with substantially reduced affinity to the native form of the laminin or laminins. Such antagonists may be used to inhibit angiogenesis.

Peptide and polypeptide antagonists can be generated by a number of techniques known to one of skill in the art. For example, a two hybrid system (e.g., Fields, S. (1989) Nature 340:245-46) would employ a fragment of a laminin as “bait” for selecting protein antagonists that bind to the laminin peptide from a library. The library of potential antagonists can be derived from a cDNA library, in one example of many possibilities. In another embodiment, the potential antagonists can be variants of known laminin-binding proteins. The genes encoding such proteins can be randomly mutagenized or subjected to gene shuffling, or other available techniques for generating sequence diversity.

Peptide and polypeptide antagonists of the invention also can be generated by techniques of molecular evolution. Libraries of genes encoding proteins can be generated by mutagenesis, gene shuffling or other available techniques for generating molecular diversity. Protein pools representing numerous variants can be selected for their ability to bind to denatured laminin, for instance by passing such protein pools over a solid matrix to which a denatured laminin has been attached. Elution with gradients of salt, for example, can provide purification of variants with affinity for the denatured laminin. A negative selection step also can be included whereby such pools are passed over a solid matrix to which native laminins have been attached. The filtrate will contain those variants within the pool that have a reduced affinity for the native form of the laminin.

Peptide and polypeptide antagonists of the invention also can be generated by phage display. A randomized peptide or protein can be expressed on the surface of a phagemid particle as a fusion with a phage coat protein. Techniques of monovalent phage display are widely available (see, e.g., Lowman H. B. et al. (1991) Biochemistry 30:10832-8.) Phage expressing randomized peptide or protein libraries can be panned with a solid matrix to which a native laminin molecule has been attached. Remaining phage do not bind native laminins, or bind native laminins with substantially reduced affinity. The phage are then panned against a solid matrix to which a denatured laminin has been attached. Bound phage are isolated and separated from the solid matrix by either a change in solution conditions or, for a suitably designed construct, by proteolytic cleavage of a linker region connecting the phage coat protein with the randomized peptide or protein library. The isolated phage can be sequenced to determine the identity of the selected antagonist.

In another embodiment, a polypeptide includes any analog, fragment or chemical derivative of a polypeptide whose amino acid residue sequence is shown herein so long as the polypeptide is an antagonist of denatured laminin, but not of native laminin. Therefore, a present polypeptide can be subject to various changes, substitutions, insertions, and deletions where such changes provide for certain advantages in its use. In this regard, a denatured laminin antagonist polypeptide of this invention corresponds to, rather than is identical to, the sequence of a recited peptide where one or more changes are made and it retains the ability to function as a denatured laminin antagonist in one or more of the assays as defined herein.

Thus, a polypeptide can be in any of a variety of forms of peptide derivatives, that include amides, conjugates with proteins, cyclized peptides, polymerized peptides, analogs, fragments, chemically modified peptides, and like derivatives.

Other Antagonists

Antagonists of the invention also can be small organic molecules, such as those natural products, or those compounds synthesized by conventional organic synthesis or combinatorial organic synthesis. Compounds can be tested for their ability to bind to a denatured laminin, for example, by using the column binding technique described above. Compounds also are selected for reduced affinity for the native form of the laminin by a similar column binding technique.

Antagonists of the invention also can be non-peptidic compounds. Suitable nonpeptidic compounds include, for example, oligonucleotides. Oligonucleotides as used herein refers to any heteropolymeric material containing purine, pyrimidine and other aromatic bases. DNA and RNA oligonucleotides are suitable for use with the invention, as are oligonucleotides with sugar (e.g., 2′ alkylated riboses) and backbone modifications (e.g., phosphorothioate oligonucleotides or peptide nucleic acids). Oligonucleotides may contain commonly found purine and pyrimidine bases such as adenine, thymine, guanine, cytidine and uridine, as well as bases modified within the heterocyclic ring portion (e.g., 7-deazaguanine) or in exocyclic positions. Oligonucleotides also encompass heteropolymers with distinct structures that also contain aromatic bases, including polyamide nucleic acids and the like.

An oligonucleotide antagonist of the invention can be generated by a number of methods known to one of skill in the art. In one embodiment, a pool of oligonucleotides is generated containing a large number of sequences. Pools can be generated, for example, by solid phase synthesis using mixtures of monomers at any or all elongation step(s). The pool of oligonucleotides is sorted by passing a solution containing the pool over a solid matrix to which a denatured laminin or fragment thereof has been affixed. Sequences within the pool that bind to the denatured laminin are retained on the solid matrix. These sequences are eluted with a solution of different salt concentration or pH. Sequences selected are subjected to a second selection step. The selected pool is passed over a second solid matrix to which native laminin has been affixed. The column retains those sequences that bind to the native laminin, thus enriching the pool for sequences specific for the denatured laminin. The pool can be amplified and, if necessary, mutagenized and the process repeated until the pool shows the characteristics of an antagonist of the invention. Individual antagonists can be identified by sequencing members of the oligonucleotide pool, usually after cloning said sequences into a host organism such as E. coli.

Disease Treatment

The present invention relates generally to the discovery that the binding of antagonists to denatured laminins but not of native laminins inhibits angiogenesis. This discovery is important because of the role that angiogenesis plays in a variety of disease processes. By inhibiting angiogenesis, one can intervene in such disease processes, ameliorate the symptoms, and in some cases cure the disease associated with the angiogenesis.

Where the growth of new blood vessels is the cause of, or contributes to, the pathology associated with a disease, inhibition of angiogenesis will reduce the deleterious effects of the disease. Examples include psoriasis, rheumatoid arthritis, diabetic retinopathy, inflammatory diseases, restenosis, macular degeneration and the like. Where the growth of new blood vessels is required to support growth of a deleterious tissue, inhibition of angiogenesis will reduce the blood supply to the tissue and thereby contribute to reduction in tissue mass based on blood supply requirements. Examples include growth of tumors where neovascularization is a continual requirement in order that the tumor grow beyond a few millimeters in diameter, and for the establishment of solid tumor metastases.

The methods of the present invention are effective in part because the therapy is highly selective for angiogenesis and not other biological processes. Only new vessel growth is inhibited by antagonists of denatured laminins, and therefore the therapeutic methods do not adversely effect mature vessels. An antagonist will bind to angiogenic sites in tumors but not to normal surrounding tissues.

There are a variety of diseases in which angiogenesis is believed to be important, referred to as angiogenic diseases, including but not limited to, inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi's sarcoma and the like cancers which require neovascularization to support tumor growth. Other suitable tumors for treatment with antagonists of the invention include melanoma, carcinoma, sarcoma, fibrosarcoma, glioma and astrocytoma. Other suitable diseases and disorders for treatment with antagonists of the invention include fibrosis, vasculitis, scleraderma, and asthma.

Some diseases associated with abnormal angiogenesis and therefore treatable by antagonists of the invention include, but are not limited to, rheumatoid arthritis, ischemic-reperfusion related brain edema and injury, cortical ischemia, ovarian hyperplasia and hypervascularity, (polycystic ovary syndrom), endometriosis, psoriasis, diabetic retinopaphy, and other ocular angiogenic diseases such as retinopathy of prematurity (retrolental fibroplastic), macular degeneration, corneal graft rejection, neuroscular glaucoma and Oster Webber syndrome.

Some examples of retinal/choroidal neuvascularization and therefore treatable by antagonists of the invention include, but are not limited to, Bests diseases, myopia, optic pits, Stargarts diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disease, systemic lupus erythematosis, retinopathy of prematurity, Eales disease, diabetic retinopathy, macular degeneration, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy.

Some examples of corneal neuvascularization and therefore treatable by antagonists of the invention include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

Methods for Inhibition of Angiogenesis

The discovery that binding of denatured laminins alone can effectively inhibit angiogenesis allows for the development of therapeutic compositions with high specificity, and therefore potentially low toxicity. Thus, although the invention discloses the use of antibody-based antagonists which have the ability to bind one or more denatured laminins, one can design or isolate other antagonists that also can specifically bind denatured laminins, but not native laminins, or with a reduced affinity for native laminins.

Prior to the discovery set forth herein, it was not known that angiogenesis, and any of the processes dependent on angiogenesis, could be inhibited in vivo by the use of reagents that antagonize cryptic epitopes in laminins, i.e., those that are found in proteolyzed or denatured laminins, but not in native forms of the same laminins.

The invention provides for a method for the inhibition of angiogenesis in a tissue, and thereby inhibiting events in the tissue which depend upon angiogenesis. Generally, the method comprises administering to the tissue a composition comprising an angiogenesis-inhibiting amount of a denatured laminin antagonist.

As described earlier, angiogenesis includes a variety of processes involving neovascularization of a tissue including “sprouting,” vasculogenesis, and vessel enlargement, all of which angiogenesis processes involve disruption of extracellular matrix collagen in blood vessels. With the exception of traumatic wound healing, corpus leuteum formation and embryogenesis, it is believed that the majority of angiogenesis processes in adults are associated with disease processes and therefore the use of the present therapeutic methods are effectively selective for these disease processes.

Methods which inhibit angiogenesis in a diseased tissue ameliorate symptoms of the disease and, depending upon the disease, can contribute to cure of the disease. In one embodiment, the invention contemplates inhibition of angiogenesis, per se, in a tissue. The extent of angiogenesis in a tissue, and therefore the extent of inhibition achieved by the present methods, can be evaluated by a variety of methods, such as are described in the Examples for detecting proteolyzed or denatured laminin-immunopositive immature and nascent vessel structures by immunohistochemistry.

As described herein, any of a variety of tissues, or organs comprised of organized tissues, can support angiogenesis in disease conditions including skin, muscle, gut, connective tissue, joints, bones and the like tissue in which blood vessels can invade upon angiogenic stimuli. Tissue, as used herein, also encompasses all bodily fluids, secretions and the like, such as serum, blood, cerebrospinal fluid, plasma, urine, synovial fluid, vitreous humor.

Thus, in one related embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

Retinal Diseases

In one embodiment, a tissue to be treated is a retinal tissue of a patient with diabetic retinopathy, macular degeneration or neovascular glaucoma and the angiogenesis to be inhibited is retinal tissue angiogenesis where there is neovascularization of retinal tissue.

Tumor Growth and Metastasis

In an additional related embodiment, a tissue to be treated is a tumor tissue of a patient with a solid tumor, a metastases, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include lung, pancreas, breast, colon, laryngeal, ovarian, Kaposi's Sarcoma and the like tissues.

Inhibition of tumor tissue angiogenesis is a particularly preferred embodiment because of the important role neovascularization plays in tumor growth. In the absence of neovascularization of tumor tissue, the tumor tissue does not obtain the required nutrients, slows in growth, ceases additional growth, regresses and ultimately becomes necrotic resulting in the death of the tumor.

Stated in other words; the present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods also are particularly effective against the formation of metastases because (1) the formation of metastases requires vascularization of a primary tumor so that the metastatic cancer cells can exit the primary tumor and (2) the establishment of metastases in a secondary site requires neovascularization to support growth of the metastases, thus both the initiation and colonization of metastases are associated with the disruption of the ECM. Thus, antagonists of the invention may have separate and additive therapeutic effects in preventing or minimizing metastasis.

In a related embodiment, the invention contemplates the practice of the method in conjunction with other therapies such as conventional chemotherapy directed against solid tumors and for control of establishment of metastases: The administration of angiogenesis inhibitor is typically conducted during or after chemotherapy, although it is preferably to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

Insofar as the present methods apply to inhibition of tumor neovascularization, the methods also can apply to inhibition of tumor tissue growth, to inhibition of tumor metastases formation, and to regression of established tumors. Antagonists of the invention will also be effective in direct treatment of metastasis as the process of metastasis necessarily requires remodeling of the extracellular matrix.

Restenosis

Restenosis is a process of smooth muscle cell (SMC) migration and proliferation at the site of percutaneous transluminal coronary angioplasty which hampers the success of angioplasty. The migration and proliferation of SMCs associated with blood vessels during restenosis is related to the process of angiogenesis which is inhibited by the present methods. Therefore, the invention also contemplates inhibition of restenosis by inhibiting angiogenic related processes according to the present methods in a patient following angioplasty procedures. For inhibition of restenosis, the denatured laminin antagonist is typically administered after the angioplasty procedure for from about 2 to about 28 days, and more typically for about the first 14 days following the procedure.

Definitions

The term “antagonist” refers to a compound that can specifically bind to a laminin or a denatured laminin under specified conditions. In preferred embodiments, specified conditions are approximately physiological conditions.

The term “binding” is meant to describe the interaction of a first compound with a second compound so that the two compounds, once they contact, typically in a solution, attach to each other in a specific fashion and remain attached in a specific fashion. However, the attachment is not necessarily irreversible. Two compounds “bind” if the interaction between the first compound and the second compound occurs with greater affinity than with respect to each other than with respect to other compounds under specified conditions. In preferred embodiments, the binding is between an antibody and an antigen.

The term “binding affinity” refers to the strength of the interaction when a first compound e.g., an antibody, that binds to a second compound, e.g., an antigen, more strongly than it binds to other compounds under specified conditions. Antagonists of the invention bind to laminin by a factor of 100 more tightly when compared to the binding of other antigens that are present in a sample when the binding takes place under specified conditions. Other antagonists of the invention may bind to laminin by a factor of 1000, by a factor of 10,000, by a factor of 100,000, or by a factor of greater than 100,000 more tightly when compared to the binding of other compounds that are present in a sample under specified conditions. Antagonists of the invention may bind to denatured laminin by a factor of 1.5, by a factor of 2, by a factor of 3, by a factor of 4, by a factor of 5, by a factor of 6, by a factor of 8, by a factor of 10, by a factor of 13, by a factor of 20, or by a factor of greater than 20 more tightly when compared to the binding of laminin in its native confirmation when the binding takes place under specified conditions. In preferred embodiments, specified conditions are approximately physiological conditions. Monoclonal antibodies of the invention comprise antibody molecules that immunoreact with isolated denatured laminin, but immunoreact with reduced affinity with the native form of the collagen. The term “reduced affinity” refers to a characteristic of antagonists of the invention that they have a greater binding affinity to laminin in a denatured state when compared their binding to laminin in its native state. In preferred embodiments, an antagonist of the invention binds to laminin in its native confirmation with about 2-fold less affinity when compared to laminin's denatured state. In other preferred embodiments, an antagonist of the invention binds to laminin in its native confirmation with about 5-fold less affinity when compared to laminin's denatured state. In further preferred embodiments, an antagonist of the invention binds to laminin in its native confirmation with about 10-fold less affinity when compared to laminin's denatured state. In still further preferred embodiments, an antagonist of the invention binds to laminin in its native confirmation with less than 10-fold affinity when compared to laminin's denatured state.

In preferred embodiments, the specified conditions of the binding is a physiological saline solution, or whole blood, or serum, or the like. In some cases, the specified conditions encompasses buffers containing detergents.

By “specific binding affinity” is meant that the antibody, or another first compound, binds to the target antigen, or target second compound, by means of its variable region with greater affinity than it binds to other antigens under specified conditions. Antibodies or antibody fragments are polypeptides that contain regions that can bind other polypeptides; in general antibodies bind specifically to their target antigens via the variable region. Antibodies may also be capable of specifically binding other antigens or proteins via domains other than the variable region; however such binding is generally associated with the antibody class. Examples of specific binding via domains other than the variable region include binding complement and Protein A. Additionally, molecules other than antibodies are also capable of binding second molecules with greater affinity than they bind to third molecules under specified conditions. Thus, in a non-limiting example, avidin binds to biotin with a greater affinity than in binds to dATP, and erbB2 binds to heregulin with a greater affinity than it binds to insulin.

The term “native laminin” may refer to both a laminin monomer or as laminin is found in situ in an organism. Denatured laminin may be obtained by thermal, enzymic, or chemical (chaotropic) means. In preferred embodiments, denatured laminin is obtained by proteolytic digestion. In further preferred embodiments, denatured laminin is obtained by MMP9 digestion.

The term “backbone domain” refers to a portion of a polypeptide's backbone chain with accompanying post-translation modifications to that region. An epitope is one embodiment of a backbone domain, but other forms are possible. In general, a backbone domain is a region of laminin or denatured laminin involved in binding to another compound, for example, an antagonist of the invention.

Antibodies or antibody fragments having specific binding affinity to a denatured laminin may be used in methods for detecting the presence and/or amount of a denatured laminin in a sample by probing the sample with the antibody under conditions suitable for denatured laminin-antibody immunocomplex formation and detecting the presence and/or amount of the antibody conjugated to the denatured laminin, then compared to binding of native laminin. Diagnostic kits for performing such methods may be constructed to include antibodies or antibody fragments specific for the denatured laminin as well as a conjugate of a binding partner of the antibodies or the antibodies themselves.

An antibody or antibody fragment with specific binding affinity to a denatured laminin can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies are well known in the art.

Antibodies having specific binding affinity to a denatured laminin may be used in methods for detecting the presence and/or amount of denatured laminin in a sample by contacting the sample with the antibody under conditions such that an immunocomplex forms and detecting the presence and/or amount of the antibody conjugated to the denatured laminin. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor. Antibody antagonists may also inhibit angiogenesis.

As used herein, an “epitope” is that amino acid sequence or sequences that are recognized or bound by an antibody or antagonist of the invention. An epitope can be a contiguous peptide sequence or can be composed of noncontiguous amino acid sequences. An antibody or antagonist can recognize or bind one or more sequences, therefore the term “epitope” can define more than one distinct amino acid sequence targets. The epitopes recognized or bound by an antibody or antagonist can be determined by peptide mapping and sequence analysis techniques well known to one of skill in the art.

Such an epitope may be a “cryptic epitope.” As used herein, a “cryptic epitope” is a laminin epitope that is not exposed for binding to an antibody or antagonist of the invention within a native laminin, but is capable of being recognized and bound by an antibody or antagonist of a denatured laminin. Peptide sequences that are not solvent exposed, or are only partially solvent exposed, in the native structure are potential cryptic epitopes. If an epitope is not solvent exposed, or only partially solvent exposed, then it is likely that the epitope is buried within the interior of the molecule. The sequence of cryptic epitopes can be identified by determining the specificity of an antibody or antagonist. Candidate cryptic epitopes also can be identified, for example, by examining the three dimensional structure of a native laminin.

The term “monoclonal antibodies” refers to antibodies that capable of recognizing and binding to a particular antigen wherein the antibodies are substantially homogenous populations. This substantially homogenous population of antibodies have substantially homogenous amino acid sequences as they are generally derived from a single immortalized cell line. They may be obtained by any technique which provides for the production of antibody molecules by continuous cell lines in culture or by bacterial production. Monoclonal antibodies may be obtained by methods known to those skilled in the art (e.g., Kohler et al., Nature 256:495-497, 1975, U.S. Pat. No. 4,376,110, and Harlow and Lane (editors), Antibodies: A Laboratory Manual, Cold Spring Harbor Laboratory (1988), all of which are hereby incorporated by reference herein in their entirety including any figures, tables, or drawings).

The term “monoclonal antibody clone” refers to an immortalized cell line that produces a substantially homogeneous population of antibodies.

In another aspect, the invention features a hybridoma which produces an antibody having specific binding affinity to a denatured laminin or a denatured laminin domain. By “hybridoma” is meant an immortalized cell line that is capable of secreting an antibody, for example an antibody to a denatured laminin of the invention. In preferred embodiments, the antibody to the denatured laminin comprises a sequence of amino acids that is able to specifically bind a denatured laminin.

Preferred monoclonal antibodies which preferentially bind to denatured laminin include monoclonal antibodies having the immunoreaction characteristics of monoclonal antibody clones LMD2, LMD9, LMD21, LMD24, LMD52, LMD105, LMD1, LMD5, LMD11, LMD17, LDM26, LMD13, LMD14, LMD15, LMD16, LMD23, LMD30, LMD209, or LMD418.

The term “polyclonal” refers to antibodies that are heterogenous populations of antibody molecules derived from the sera of animals immunized with an antigen or an antigenic functional derivative thereof. The term polyclonal refers to the fact that such a population of antibodies arises from more than one clone of antibody producting cell, and therefore, as a group the antibodies are expected to have divergent amino acid sequences, especially in the variable region. Additionally, different classes of antibodies, e.g., IgG or IgM, may be produced and included with a given sample of polyclonal antibodies. For the production of polyclonal antibodies, various host animals may be immunized by injection with the antigen. Various adjuvants may be used to increase the immunological response, depending on the host species.

Antibodies of the invention may be “humanized” by grafting the complementarity-determining regions (CDRs) or variable regions, into the framework of a human immunoglobulin molecule of a certain class, such as an IgG molecule. This produces a monoclonal antibody that is antigenically virtually identical to a human immunoglobulin but binds to the same antigen as the original antibody CDR region. Thus, these recombinant antibodies can be used in treatments in humans with far less risk of complications.

Other modifications to antibodies, or other antagonists, encompassed by the instant invention include, genetic fusion proteins with other biologically active proteins or ligands or part of a bi-specific antibody where two antibodies are linked together.

The term “antibody fragment” refers to a portion of an antibody, such as the hypervariable region and portions of the surrounding heavy and light chains, or the CDR region, that displays specific binding affinity for a particular molecule. A hypervariable region is a portion of an antibody that physically binds to the polypeptide target. The term “CDR region” of an antibody (complementarity determining region) refers to the antigen-binding region of an antibody. The paratope, or antibody combining site is that structural portion of an antibody molecule comprised of heavy and light chain variable regions that specifically binds antigens.

Antibody fragments of the invention contain the paratope, and include those portions known in the art as Fab, Fab′, F(ab′)₂ and F(v). The Fab fragment lacks the Fc receptor, is soluble and affords therapeutic advantage in serum half life, and diagnostic advantages in modes of using the soluble Fab fragment. For example, Fab and F(ab′)₂ portions (fragments) of antibodies are prepared by the proteolytic reaction of papain and pepsin, respectively, on substantially intact antibodies by methods that are well known. See for example, U.S. Pat. No. 4,342,566. Fab′ antibody portions also are well known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact immunoglobulin molecules are preferred.

Antibodies or antibody fragments having specific binding affinity to a denatured laminin may be used in methods for detecting the presence and/or amount of a denatured laminin in a sample by probing the sample with the antibody under conditions suitable for denatured laminin-antibody immunocomplex formation and detecting the presence and/or amount of the antibody conjugated to the denatured laminin. Diagnostic kits for performing such methods may be constructed to include antibodies or antibody fragments specific for the denatured laminin as well as a conjugate of a binding partner of the antibodies or the antibodies themselves. Such kits will also include one or more negative control antibodies.

The term “negative control antibody” refers to an antibody derived from similar source as the antibody having specific binding affinity, but where it displays no binding affinity to a denatured laminin, or the same binding affinity to a denatured laminin as to a laminin.

An antibody or antibody fragment with specific binding affinity to a denatured laminin can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies are well known in the art.

Antibody antagonists of the invention may be generated according to a number of methods knosn to one skilled in the art. Antibodies thus generated can be selected for their ability to bind to denatured laminin and for a reduced affinity for the native form of the same laminin. Antibodies can, for example, be generated by the method of “subtractive immunization. (see, e.g., Brooks et al., (1993) J. Cell Biol. 122:1351-59).

The terms “protein,” “polypeptide,” and “peptide” are used herein interchangeably and are used in their conventional meaning, i.e., a chain of amino acids. None of the terms imply any set length or range of lengths of amino acid chains. These terms also do not imply or exclude post-translational (or post-synthetic) modifications of the polypeptide, e.g., glycosylations, acetylations, phosphorylations, and the like, as well as other modifications known to those of skill in the art, both naturally occurring and non-naturally occurring. A polypeptide may be an entire protein, or a region of the amino acid chain. A protein may comprise more than one polypeptide.

The term “linear peptide” is used herein to denote a conventional peptide backbone that stretches from the amino terminus, which is free from attachment via a peptide bond to another amino acid, to the carboxy terminus, which is likewise free from attachment via a peptide bond to another amino acid. Note that the amino acid which is located at the amino terminus of a linear peptide is connected to the peptide backbone only via the carboxy portion of the amino acid; and the amino acid which is located at the carboxy terminus of the peptide chain is bound to the chain only by a peptide bond formed by the alpha amino group.

The term “cyclic peptide” is used herein to denote a peptide backbone that does not have an amino terminus, nor a carboxy terminus. That is, all the amino acids contained in the backbone are bound via a peptide bond to the alpha carbon to two other amino acids that are also in the backbone.

The term “non-peptidic compound” includes such non-limiting examples as carbohydrates, lipids, synthetic polymers, etc.

The term “oligonucleotide” refers to a relatively short chain of polynucleotides which may contain between about 10 and about 2000 nucleotides. Such a term also refers to all forms of DNA and all forms of RNA. Often the term refers to single-stranded deoxyribonucleotides, but it may also refer as well to single- or double-stranded ribonucleotides, RNA:DNA hybrids, double-stranded DNAs, and peptide nucleic acids, among others. The term “oligonucleotide” may include chains which are at least as long as 10, 20, 30, 40, 50, 70, 80, 100, 120, 150, 175, and 200 nucleotides. These chains may be up to 2000, 1500, 1000, 800, 600, 500, 450, 400, 350, 300, 250, 200, 175, 150, 125 and 100 nucleotides in length. Thus, for example, the oligonucleotides may be between 150 and 600 nucleotides in length.

Oligonucleotides, especially single-stranded DNA oligonucleotides, are often synthesized by chemical methods, such as those implemented by automated synthesizers. However, oligonucleotides can be made by a variety of other methods, including in vitro recombinant DNA-mediated techniques and by expression of RNAs or DNAs in cells organisms or in vitro.

Initially, chemically synthesized DNAs typically are obtained without a 5′ phosphate, therefore are frequently phosphorylated before further use. The 3′ end of a chemically synthesized oligonucleotide generally has a free hydroxyl group and may be readily ligated without further modification.

The term “structural modification” or “nucleic acid structural modification” refers to any molecular structural change to any nucleic acid base moiety, nucleoside moiety, or nucleotide moiety of any form of RNA or DNA nucleic acid. Structural modifications can consist of post-transcriptional modifications, derivatives, reactions to produce natural products, or cellular chemistries or reactions that result in a molecular structural change that modifies the base, nucleoside, or nucleotide moieties of a nucleic acid. Structural modifications include, but are not limited to, for example, various alkylations, methylations, thiolations, oxidations, peptide derivatizations, sugar derivatizations, and radiation-induced or radical-induced reactions of nucleic acid bases, nucleosides, or nucleotides.

The term “conjugate” as used herein is defined as the tethering, binding or association of a molecule with/to another molecule, composition, compound, or detectable label. Such tethering, binding or association includes covalent interactions or non-covalent interactions, such as biotin/avidin.

The term “cytotoxic agent” as used herein refers to a molecule, compound, toxin, composition, or biological entity which is used to kill a cell. In a preferred embodiment the cell is a tumor cell. “Cytotoxic agents” refer to compounds which cause cell death primarily by interfering directly with the cell's functioning or inhibit or interfere with cell meiosis. Examples of such agents include alkylating agents, tumor necrosis factors, intercalators, microtubulin inhibitors, and topoisomerase inhibitors.

The term “cytostatic agent” as used herein refers to a molecule, compound, toxin, composition, or biological entity which is used to prevent a cell from further division. In a preferred embodiment the cell is a tumor cell. “Cytostatic agents” refer to compounds which interfere with a cell's growth and division primarily by interfering directly with the cell's functioning, the cell's metabolism, or inhibit or interfere with cell meiosis.

The term “inhibiting” refers to decreasing the cellular activity and the extracellular activity associated with a given process. Thus, the overall rate of a process slows or perhaps is eliminated entirely. The term “inhibiting” may also refer to decreasing the cellular or the extracellular activity of a polypeptide by decreasing the interaction of a polypeptide with its natural binding partner; preferably this inhibits the catalytic activity of a polypeptide.

The term “tissue” as used herein can refers to any tissue in a biological organism. Such tissues may include, but are not limited to, lung, heart, blood, liver, muscle, brain, pancreas, skin, and others. The invention also relates to administering a compound to a tissue by increasing the concentration of the antagonist of the invention in a biological fluid that bring the antagonist in proximity to the tissue to be treated. The fluids may include, but are not limited to, blood, serum, plasma, tears, saliva, milk, urine, amniotic fluid, semen, plasma, oviductal fluid, and synovial fluid.

The term “organ” relates to any organ in an organism, isolated from an organism or any portion of an organ. Examples of organs and tissues are neuronal tissue, brain tissue, spleen, heart, lung, gallbladder, pancreas, testis, ovary, and kidney. These examples are not limiting and the invention relates to any organ and any tissue from an organism or isolated from an organism.

The terms “administration” or “administering” refer to a method of incorporating a compound into the cells or tissues of an animal, preferably a mammal in order to treat or prevent an abnormal condition. When the compound or prodrug of the invention is provided in combination with one or active agents, the terms “administration” “administering” include sequential or concurrent introduction of the compound or prodrug with the other agent(s). For cells harbored within the organism, many techniques exist in the art to administer compounds, including (but not limited to) oral, injection, parenteral, dermal, and aerosol applications.

The effect of administering a compound on organism function can then be monitored. The organism is preferably a mouse, rat, rabbit, guinea pig, or goat, more preferably a monkey or ape, and most preferably a human.

The term “chemotherapy” refers to any one of a number of treatment regimes; the term is primarily, but not exclusively, used in connection of treating cancers using drugs that are selectively toxic for the cancerous cells. Thus, “chemotherapy” implies a series of doses of one or more chemotherapeutic agents under the direction of a treating physician. Frequently, the administration of chemotherapeutic agents is accompanied by significant side effects that need to be monitored by the treating physician.

The term “chemotherapeutic agent” as used herein includes, for example, hormonal agents, antimetabolites, DNA-interactive agents, tubulin-interactive agents, and others, such as asparaginase or hydroxyurea. Each of these types of chemotherapeutic agents may be further subdivided. Possible chemotherapeutic agents may be selected from these groups or from other groups. For a detailed discussion of the chemotherapeutic agents and their method of administration, see Dorr, et al., Cancer Chemotherapy Handbook, 2d edition, Appleton & Lange (Connecticut, 1994) herein incorporated by reference. See also U.S. Pat. No. 6,316,462 for a listing of potential chemotherapeutic agents.

The term “radiation therapy” refers to any one of a number of treatment regimes; the term is primarily, but not exclusively, used in connection of treating cancers. Thus, “radiation therapy” implies exposing a patient to a series of doses of radiation or radioisotopes under the direction of a treating physician. Frequently, the administration of radiation therapy is accompanied by significant side effects that need to be monitored by the treating physician.

The term “heating the tissue” refers to elevating the temperature of a region of tissue, usually in vivo. This may be accomplished by application of a heat source, or by radiation such as microwave radiation.

The term “detecting” as used herein refers to any method of verifying the presence of a given molecule, event, or series of events. The techniques used to accomplish this may include, but are not limited to, PCR, sequencing, PCR sequencing, molecular beacon technology, hybridization, and hybridization followed by PCR. Examples of reagents which might be used for detection include, but are not limited to, radiolabeled probes, enzymatic labeled probes (horseradish peroxidase, alkaline phosphatase), and affinity labeled probes (biotin, avidin, or streptavidin).

The term “sample” as used herein refers to an aliquot of material, very frequently an aqueous solution or an aqueous suspension derived from biological material. Samples to be assayed for the presence of an analyte by the methods of the present invention include, for example, cells, proteins extracted from cells, and include human and animal body fluids such as whole blood, serum, plasma, cerebrospinal fluid, sputum, bronchial washing, bronchial aspirates, urine, lymph fluids and various external secretions of the respiratory, intestinal and genitourinary tracts, tears, saliva, milk, white blood cells, myelomas and the like; biological fluids such as cell culture supernatants; tissue specimens which may be fixed; and cell specimens which may be fixed. The samples used in the above-described methods will vary based on the assay format, and the nature of the tissues, cells or extracts to be assayed. Methods for preparing protein extracts from cells or samples are well known in the art and can be readily adapted in order to obtain a sample that is compatible with the method of the instant invention.

The term “organism” refers to any living creature capable of reproduction. Any organism may be treated by the antagonists of the invention. In preferred embodiments, the organism is a mammal. The term “mammal” refers preferably, but is not limited to, to such organisms as rodents, ungulates, primates, mice, rats, rabbits, guinea pigs, horses, sheep, pigs, goats, and cows, more preferably to cats, dogs, monkeys, and apes, and most preferably to humans. The term “mammalian” or “mammal” as used herein may also refer to any warm-blooded animal such as is described above or cell derived from that animal.

The term “contacting” as used herein refers to adding together a solution or composition comprising an antagonist of the invention, or another compound, with a liquid medium bathing the cells, tissue or organ from an organism. Alternately, “contacting” may refer to mixing together a solution or composition comprising an antagonist of the invention, or another compound, with a liquid such as blood, serum, or plasma derived from an organism. The solution comprising the compound may also comprise another component, such as dimethyl sulfoxide (DMSO). DMSO facilitates the uptake of the compounds or solubility of the compounds. The solution comprising the test compound may be added to the medium bathing the cells, tissues, or organs, or mixed with another liquid such as blood, by utilizing a delivery apparatus, such as a pipette-based device or syringe-based device.

The term “screening” refers to a method of comparing the activity of individual compounds selected from within a large field of compounds, such as small organic molecules. After an assay is established in the laboratory, the ability of a compound or a small set of compounds selected from a set of compounds are individually or collectively assessed in their ability to modulate the activity demonstrated in the assay. In preferred embodiments, compounds are assayed for their ability to bind denatured laminin, and this binding is compared subsequently to their ability to bind native laminin. Compounds may be screened one at a time in each assay, or small sets of compounds may be mixed together and assayed simulateously in one reaction container. Such screening assays commonly are conducted in 96-well plates, or the like. Compounds to be screened may be derived from combinatorial libraries. Compounds to be screened may also be derived from rational drug design methods.

The compounds to be screened include, but are not limited to, extracellular, intracellular, biological or chemical origin. One skilled in the art can measure the change in the activity of the reporter assay appears after the cell has been exposed to a test compound(s). One skilled in the art could, for example, measure the increase or decrease in the rate of angiogenesis using an assay as described herein, or another assay. of a cell's ability to produce the fluorescent product of the luciferase protein. Alternatively, one skilled in the art can examine the increase or decrease of expression of marker genes associated with angiogenesis or another process under study. a cell's ability to produce the fluorescent product of the luciferase protein.

The term “modulator” refers to a compound which can alter the course or sequence of events associated with a process such as angiogenesis or inflammation. A modulator preferably reduces the rate a process occurs depending on the concentration of the compound provided to the tissue, more preferably reduces or increases the rate of a process depending on the concentration of the compound provided to the tissue, or most preferably increases the rate a process occurs depending on the concentration of the compound provided to the tissue. In other preferred embodiments, a modulator increases the rate a polypeptide is modified, or inhibits the rate a polypeptide is modified depending on the concentration of the compound exposed to the polypeptide. In still further preferred embodiments, a modulator increases the rate a polypeptide associates with its natural binding partner or inhibits the rate a polypeptide associates with its natural binding partner depending on the concentration of the compound exposed to the polypeptide.

The term “treating” refers to administering a composition having a therapeutic effect and at least partially alleviating or abrogating an abnormal condition in the organism. Note that the treatment need not provide a complete cure and will be considered effective if at least one symptom is improved or eradicated. Furthermore, the treatment need not provide a permanent improvement of the medical condition or other abnormal conditions, although this is preferable.

The term “treating cancer” refers to administrating a composition to a mammal afflicted with a cancerous condition and refers to an effect that alleviates the cancerous condition by killing the cancerous cells, inhibiting the growth of the cancerous cells, and/or inhibiting the process of metastasis of the cancer, including angiogenesis.

The term “preventing” refers to decreasing the probability that an organism contracts or develops an abnormal condition.

An antagonist of the invention can be further modified with a modifying group. Modifying groups may include groups comprising biochemical labels or structures, such as biotin, fluorescent-label-containing groups, light scattering or plasmon resonant particle, a diethylenetriaminepentaacetyl group, a (O)-menthoxyacetyl group, a N-acetylneuraminyl group, a cholyl structure or an iminobiotinyl group. An antagonist of the invention may be modified at its carboxy terminus with a cholyl group according to methods known in the art. Cholyl derivatives and analogs may also be used as modifying groups. For example, a preferred cholyl derivative is Aic (3-(O-aminoethyl-iso)-cholyl), which has a free amino group that can be used to further modify the peptide antagonist of the invention. A modifying group may be a “biotinyl structure,” which includes biotinyl groups and analogues and derivatives thereof (such as a 2-iminobiotinyl group). In another embodiment, the modifying group may comprise a fluorescent-label group, e.g., a fluorescein-containing group, such as a group derived from reacting a antagonist of the invention derived peptidic structure with 5-(and 6-)-carboxyfluorescein, succinimidyl ester or fluorescein isothiocyanate. The antagonist of the invention may also be modified by attaching other fluorescent labels including rhodamine, dichlorotriazinylamine fluorescein, dansyl chloride or phycoerythrin and energy transfer fluorescent dyes or fluorescent ion indicators. In various other embodiments, the modifying group(s) may comprise an N-acetylneuraminyl group, a trans-4-cotininecarboxyl group, a 2-imino-1-imidazolidineacetyl group, an (S)-(−)-indoline-2-carboxyl group, a (−)-menthoxyacetyl group, a 2-norbornaneacetyl group, a γ-oxo-5-acenaphthenebutyryl, a (−)-2-oxo-4-thiazolidinecarboxyl group, a tetrahydro-3-furoyl group, a 2-iminobiotinyl group, a diethylenetriaminepentaacetyl group, a 4-morpholinecarbonyl group, a 2-thiopheneacetyl group or a 2-thiophenesulfonyl group. In other embodiments, light scattering groups, magnetic groups, nanogold, other proteins, a solid matrix, radiolabels, or carbohydrates may be attached.

In still other aspects, the modifying group may be an oligomer, for example, polyethylene glycol, an oligonucleotide, a polypeptide (which may or may not be derived from the original peptide antagonist of the invention) or one moiety of a binding pair.

An antagonist of the invention may be further modified to alter the specific properties of the compound while retaining the desired functionality of the compound. For example, in one embodiment, the compound may be modified to alter a pharmacokinetic property of the compound, such as in vivo stability, solubility, bioavailability or half-life. The compound may be modified to label the compound with a detectable substance. The compound may be modified to couple the compound to an additional therapeutic moiety. To further chemically modify the compound, such as to alter its pharmacokinetic properties, reactive groups can be derivatized. For example, when the modifying group is attached to the amino-terminal end of the peptide antagonist of the invention's core domain, the carboxy-terminal end of the compound may be further modified. Potential C-terminal modifications include those that reduce the ability of the compound to act as a substrate for carboxypeptidases. Examples of C-terminal modifiers include an amide group, an ethylamide group and various non-natural amino acids, such as D-amino acids, β-alanine, C-terminal decarboxylation, and a C-terminal alcohol. Alternatively, when the modifying group is attached to the carboxy-terminal end of the aggregation core domain, the amino-terminal end of the compound may be further modified, for example, to reduce the ability of the compound to act as a substrate for aminopeptidases.

An antagonist of the invention may be modified by the addition of polyethylene glycol (PEG). PEG modification may lead to improved circulation time, improved solubility, improved resistance to proteolysis, reduced antigenicity and immunogenicity, improved bioavailability, reduced toxicity, improved stability, and easier formulation (For a review see, Francis et al., International Journal of Hematology 68:1-18, 1998). PEGylation may also result in a substantial reduction in bioactivity.

An antagonist of the invention may also be coupled to a radioisotope such as yttrium-90 or iodine-131 for therapeutic purposes (see, e.g., DeNardo et al., “Choosing an optimal radioimmunotherapy dose for clinical response,” Cancer 94(4 Suppl): 1275-86, 2002; Kaltsas et al., “The value of radiolabelled MIBG and octreotide in the diagnosis and management of neuroendocrine tumours,” Ann Oncol 12 Suppl 2:S47-50,2001).

An antagonist of the invention can be further modified to label the compound by reacting the compound with a detectable substance. In some aspects of the invention, suitable detectable substances include various enzymes, prosthetic groups, fluorescent materials, luminescent materials, light scattering or plasmon resonant materials, and radioactive materials. Examples of suitable enzymes include horseradish peroxidase, alkaline phosphatase, beta-galactosidase, or acetylcholinesterase. Examples of suitable prosthetic groups which are members of a binding pair and are capable of forming complexes include streptavidin/biotin, avidin/biotin and an antigen/antibody complex (e.g., rabbit IgG and anti-rabbit IgG). Examples of suitable fluorescent materials include umbelliferone, fluorescein, fluorescein isothiocyanate, rhodamine, dichlorotriazinylamine fluorescein, dansyl chloride or phycoerythrin and energy transfer fluorescent dyes. An example of a luminescent material includes luminol. Examples of light scattering or plasmon resonant materials include gold or silver particles and quantum dots. Examples of suitable radioactive material include ¹⁴C, ¹²³I, ¹²⁴I, ¹²⁵I, ¹³¹I, Tc99m, ³⁵S or ³H. An antagonist of the invention may be radioactively labeled with ¹⁴C, either by incorporation of ¹⁴C into the modifying group or one or more amino acid structures in the antagonist of the invention. Labeled antagonist of the invention may be used to assess the in vivo pharmacokinetics of the compounds, as well as to detect disease progression or propensity of a subject to develop a disease, for example for diagnostic purposes. Tissue distribution denatured laminin can be detected using a labeled antagonist of the invention either in vivo or in an in vitro sample derived from a subject. For use as an in vivo diagnostic agent, an antagonist of the invention may be labeled with radioactive technetium or iodine. A modifying group can be chosen that provides a site at which a chelation group for the label can be introduced, such as the Aic derivative of cholic acid, which has a free amino group. For example, a tyrosine residue within a peptide antagonist of the invention sequence may be substituted with radioactive iodotyrosyl. Any of the various isotopes of radioactive iodine may be incorporated to create a diagnostic or therapeutic agent. ¹²³I (half-life=13.2 hours) may be used for whole body scintigraphy, ¹²⁴I (half life=4 days) may be used for positron emission tomography (PET), ¹²⁵I (half life=60 days) may be used for metabolic turnover studies and ¹³¹I, (half life=8 days) may be used for whole body counting and delayed low resolution imaging studies.

In an alternative chemical modification, an antagonist of the invention may be prepared in a “prodrug” form, wherein the compound itself does not act as a an antagonist of denatured laminin, but rather is capable of being transformed, upon metabolism in vivo, into an antagonist of denatured laminin as defined herein. For example, in this type of compound, the modifying group can be present in a prodrug form that is capable of being converted upon metabolism into the form of an active antagonist of denatured laminin. Such a prodrug form of a modifying group is referred to herein as a “secondary modifying group.” A variety of strategies are known in the art for preparing peptide prodrugs that limit metabolism in order to optimize delivery of the active form of the peptide-based drug.

Treatment

The present method for inhibiting angiogenesis in a tissue, and therefore for also practicing the methods for treatment of angiogenesis-related diseases, comprises contacting a tissue in which angiogenesis is occurring, or is at risk for occurring, with a composition comprising a therapeutically effective amount of a denatured laminin antagonist capable of binding to denatured or proteolyzed laminin, but with less affinity to native forms of laminin. Thus, the method comprises administering to a patient a therapeutically effective amount of a physiologically tolerable composition containing an denatured laminin antagonist of the invention.

The dosage ranges for the administration of the denatured laminin antagonist depend upon the form of the antagonist, and its potency, as described further herein, and are amounts large enough to produce the desired effect in which angiogenesis and the disease symptoms mediated by angiogenesis are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage also can be adjusted by the individual physician in the event of any complication.

When the compounds of the present invention are administered to a subject, they are prepared as therapeutic compositions. Such compositions may routinely contain pharmaceutically acceptable concentrations of salts, buffering agents, preservatives, compatible carriers, and optionally other therapeutic agents. The therapeutic composition may be administered by any conventional route, including injection or by gradual infusion over time. The administration may, depending on the composition being administered, for example, be oral, pulmonary, intravenous, intraperitoneal, intramuscular, intracavity, subcutaneous, or transdermal.

The term “therapeutic composition” is used interchangeably with the terms “active compound,” “active agent” or “active composition” and as used herein refers to any of the denatured laminin antagonists of the invention which produce a biological effect, e.g., the inhibition of angiogenesis. The therapeutic compositions used in the present invention preferably are sterile and contain a therapeutically effective amount of a therapeutic composition for producing the desired response in a unit of weight or volume suitable for administration to a patient.

The compositions are administered in therapeutically effective amounts. The term “therapeutically effective amount” as used herein means that amount of active compound, prodrug or pharmaceutical agent that elicits a biological or medicinal response in a tissue, system, animal or human that is being sought by a researcher, veterinarian, medical doctor or other clinician in order to provide a therapeutic effect.

The terms “administration” or “administering” refer to a method of incorporating a compound into the cells or tissues of an animal, preferably a mammal in order to treat or prevent an abnormal condition. When the compound or prodrug of the invention is provided in combination with one or active agents, the terms “administration” “administering” include sequential or concurrent introduction of the compound or prodrug with the other agent(s). For cells harbored within the organism, many techniques exist in the art to administer compounds, including (but not limited to) oral, injection, parenteral, dermal, and aerosol applications.

The effect of administering a compound on organism function can then be monitored. The organism is preferably a mouse, rat, rabbit, guinea pig, or goat, more preferably a monkey or ape, and most preferably a human.

The term “therapeutic effect” refers to the inhibition or activation of factors causing or contributing to the abnormal condition (including a disease or disorder such as metastasis). A therapeutic effect relieves or prevents to some extent one or more of the symptoms of the abnormal condition. In reference to the treatment of abnormal conditions, a therapeutic effect can refer to one or more of the following: (a) an increase or decrease in the proliferation, growth, and/or differentiation of cells; (b) inhibition (i.e., slowing or stopping) or acceleration of cell death; (c) relieving, to some extent, one or more of the symptoms associated with an abnormal condition; (d) enhancing or inhibiting the function of the affected population of cells; (e) activating an enzyme activity present in cells associated with the abnormal condition; and (f) inhibiting an enzyme activity present in cells associated with the abnormal condition.

The term “abnormal condition” refers to a function in the cells or tissues of an organism that deviates from their normal functions in that organism and includes, but is not limited to, conditions commonly referred to as diseases or processes associated with diseases. An abnormal condition can relate to angiogenesis, cell proliferation, cell differentiation, remodeling of the extracellular matrix, cell survival, cellular function, or the activities of enzymes within a cell. Abnormal conditions relating to cell proliferative disorders include cancers, fibrotic and mesangial disorders, abnormal angiogenesis and vasculogenesis, wound healing, psoriasis, diabetes mellitus, and inflammation. Abnormal conditions relating to differentiation include, but are not limited to, neurodegenerative disorders, slow wound healing rates, and slow tissue grafting healing rates. Abnormal conditions relating to cell survival refer to conditions in which programmed cell death (apoptosis) pathways are activated or abrogated. A number of extracellular matrix proteins are associated with the angiogenesis.

The term “aberration,” in conjunction with the function of laminin molecules, refers to a laminin molecule that it no longer interacts correctly with other elements of the extra-cellular matrix, is conformationally changed such that it can no longer interact with a natural binding partner, is no longer modified by another protein, or no longer interacts with a natural binding partner.

In one embodiment, a therapeutically effective amount is an amount of denatured laminin antagonist sufficient to produce a measurable inhibition of angiogenesis in the tissue being treated, i.e., an angiogenesis-inhibiting amount. Inhibition of angiogenesis can be measured in situ by immunohistochemistry, as described herein, or by other methods known to one skilled in the art. Potency of a denatured laminin antagonist can be measured by a variety of means including inhibition of angiogenesis in the CAM assay, in the in vivo rabbit eye assay, in the in vivo chimeric mouse:human assay and in like assays.

A therapeutically effective amount of a denatured laminin antagonist of this invention in the form of a monoclonal antibody is typically an amount such that when administered in a physiologically tolerable composition is sufficient to achieve a plasma concentration of from about 0.01 microgram (μg) per milliliter (mL) to about 100 μg/mL, preferably from about 1 μg/mL to about 5 μg/mL, and usually about 5 μg/mL. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

Where the antagonist is in the form of a fragment of a monoclonal antibody, the amount can readily be adjusted based on the mass of the fragment relative to the mass of the whole antibody. A preferred plasma concentration in molarity is from about 2 micromolar (μM) to about 5 millimolar (mM) and preferably about 100 μM to 1 mM antibody antagonist.

A therapeutically effective amount of a denatured collagen antagonist of this invention in the form of a polypeptide, or small molecule, is typically an amount of polypeptide such that when administered in a physiologically tolerable composition is sufficient to achieve a plasma concentration of from about 0.1 microgram (μg) per milliliter (mL) to about 200 μg/mL, preferably from about 1 μg/mL to about 150 μg/mL. Based on a polypeptide having a mass of about 500 grams per mole, the preferred plasma concentration in molarity is from about 2 micromolar (μM) to about 5 millimolar (mM) and preferably about 100 μM to 1 mM polypeptide antagonist. Stated differently, the dosage per body weight can vary from about 0.1 mg/kg to about 300 mg/kg, and preferably from about 0.2 mg/kg to about 200 mg/kg, in one or more dose administrations daily, for one or several days.

The amount may be adjusted by the health practitioner. Factors within the knowledge and expertise of the health practitioner include: the particular condition being treated, the severity of the condition, the patient's age, the patient's physical condition, the patient's weight, the duration of the treatment, the nature of concurrent therapy (if any), the route of administration and like factors. These factors are well known to those of ordinary skill in the art and can be addressed with no more than routine adjustments. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for other reasons. The response of the patient can, for example, be measured by determining the amount of circulating laminin following administration of the therapeutic composition, or by measuring the rate of angiogenesis. Other assays will be known to one of ordinary skill in the art and can be employed for measuring the level of the response.

The doses of the active compounds administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses via a different delivery route) may be employed to the extent that patient tolerance permits.

Other protocols for the administration of therapeutic compositions will be known to one of ordinary skill in the art, in which the dose amount, schedule of injections, sites of injections, mode of administration and the like vary from the foregoing. Administration of therapeutic compositions to mammals other than humans, e.g. for testing purposes or veterinary therapeutic purposes, is carried out under substantially the same conditions as described above.

The monoclonal antibodies, peptides, or other antagonists of the invention can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, antagonists including monoclonal antibodies, polypeptides, and derivatives thereof can be administered intravenously, intraperitoneally, intramuscularly, subcutaneously, intracavity, transdermally, topically, intraocularly, orally, intranasally, or intraarticularly; and can be delivered by peristaltic means.

The therapeutic compositions containing the monoclonal antibodies, peptides, or other antagonists of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., a carrier, or a vehicle.

In one preferred embodiment, the denatured laminin antagonist is administered in a single dosage intravenously.

The compositions of the instant invention are administered in a manner compatible with the dosage formulation, and in a therapeutically effective amount. The quantity to be administered and timing depends on the patient to be treated, capacity of the patient's system to utilize the active ingredient, and degree of therapeutic effect desired. Precise amounts of active ingredient required to be administered depend on the judgement of the practitioner and are peculiar to each individual. However, suitable dosage ranges for systemic application are disclosed herein and depend on the route of administration. Suitable regimes for administration also are variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The patient treated in the present invention in its many embodiments is desirably a human patient, although it is to be understood that the principles of the invention indicate that the invention is effective with respect to all mammals, which are intended to be included in the term “patient.” In this context, a mammal is understood to include any mammalian species in which treatment of diseases associated with angiogenesis is desirable, particularly agricultural and domestic mammalian species. Such a patient can be, for example, a pig, a cow, a horse, a goat, a sheep, a mule, a donkey, a dog, a cat, a rabbit, a guinea pig, a monkey or ape, a mouse or a rat.

### EXAMPLES

### Example 1

### Obtaining the Antibodies

Use subtractive immunization to isolate a set of monoclonal antibodies that react specifically with the denatured forms of human laminin. The subtractive immunization technique experimentally manipulates the immune response of a mouse so that one can selectively enhance the generation of antibodies to rare and low abundant epitopes among a mixture of common highly antigenic epitopes. Briefly, groups of six female BALB/c mice are injected I.P. with 200 μg of triple helical human native laminin, respectively. Twenty-four and forty-eight hours following the injections of the native antigens, the mice are injected (I.P.) with the tollerizing agent cyclophosphamide (150 mg/kg). The tollerizing agent kills activated B-cells that produce antibodies against common determinants within the native antigens. The tollerization is repeated three times to reduce any immune response to common epitopes within native antigens. Twenty-five days after the last tollerization injection, the mice are injected with thermally denatured Type-V collagen, fibronectin, or laminin to stimulate immune responses to epitopes exposed by denaturation. The denatured antigens are injected every three weeks for a total of four injections. Serum from each mouse is tested for immunoreactivity with native and denatured antigens by ELISA. All mice demonstrating high immunoreactivity specifically to the denatured antigens are used for the production of hybridomas.

Preparation of antigens: Purified human laminin is purchased from Sigma (St. Louis, Mo.). The native proteins are diluted in sterile PBS for antigen injections. For thermal denaturization, the proteins are heated in a boiling water bath for 30 min, and rapidly cooled in an ice water bath.

Testing of serum reactivity to denatured/degraded ECM proteins using ELISA: Serum from each mouse are tested for immunoreactivity to each of the native and thermally denatured antigens by ELISA. Microtiter plates are coated overnight at 4° C. with Type-V collagen, laminin and fibronectin (native and thermally denatured) at a concentration of 25 μg/ml. The wells are then blocked with protease-free BSA (100 μg/ml) in PBS for 4 hr. Sera or monoclonal antibodies are incubated at appropriate titers for 2 hr. at 37° C., followed by incubation with goat anti-mouse IgG and IgM (H+ L) conjugated with peroxidase (1:3,000 dilution) for 2 hr. Finally, OPD (0.4 mg/ml) in 80 mM citrate-phosphate buffer (pH 5.0) are added into the washed plates to quantify the bound antibodies. The chromagenic reaction is stopped by 25 μl of 4N H₂SO₄. Absorbance at 490 nm is determined, and optical values were corrected for nonspecific receptor binding to BSA. The assay is performed in triplicates. All mice showing reactivity for the denatured antigens are used for hybridoma production.

Preparation of hybridoma: Hybridoma fusions are carried out by standard techniques as described previously (Fazekas, d. S., Groth, S., and Scheidegger, D. (1980) J. Immunol. Methods 35, 1-21). Briefly, the spleens and lymph nodes are removed from immunized mice and washed in 10 ml of DMEM. Spleen cells are harvested from the tissues by standard ballooning techniques. Recovered cells are washed in DMEM twice. Erythrocytes are eliminated by incubation in 0.1 M NH₄Cl for 10 minutes. The spleen cells then are washed and mixed with myeloma cells at a ratio of 4:1 (spleen cells:myeloma). PEG-4000 (0.5 ml) is added to cells slowly over a 60 sec period with gentle mixing and incubated for 90 sec at 37° C. The cells are subsequently rinsed with 20 ml of sterile saline. The fused cells are resuspended in complete HAT medium and are subjected to 96-well microtiter plates at a concentration of 2×10⁵ cells per well. The hybridoma cultures are allowed to grow undisturbed for 7 to 14 days. Supernatants from the resulting hybridomas are screened for reactivity by ELISA. Pools of hybridomas are stored in liquid nitrogen until the time of cloning. All hybridomas are cloned twice by limiting dilution to assure clonality.

Isotyping of monoclonal antibodies produced by hybridomas: Monoclonal antibodies demonstrating preferential reactivity for denatured ECM proteins are isotyped according to established procedures utilizing the Mouse Typer Sub-Isotyping Kit (Bio-Rad) according to the manufacturer's protocol.

Purification of monoclonal antibodies: IgG monoclonal antibodies are purified from culture media or ascites by affinity to Protein-A-Agarose using standard techniques. IgM monoclonal antibodies are purified by affinity to Mannin-Binding-Column (Pierce) using the protocol provided by the manufacturer.

To ensure the productivity, more animals than normal are used (6 mice). In addition, those monoclonal antibodies with the desired immunoreactivity but low titer are not excluded from further analysis. With these modifications, for each denatured antigen, 30-40 positive hybridomas are expected; some of these positive hybridomas should display antiangiogenic activity (approximately 4-8).

Limitations and alternatives: Several novel antiangiogenic monoclonal antibodies are identified. As cyclophosphamide is an immunosuppressant, it may suppress B-cell proliferation so that few viable B-cell remain. If this occurs, the dosage of cyclophosphamide can be reduced to a lower level (e.g. 100 mg/kg). Alternate approaches, such as immunization with specific peptides that are suspected to play a role in regulating angiogenesis, may be used to identify antibodies that recognize cryptic sites.

### Example 2

### Obtaining the Antibodies

Subtractive immunization is used to isolate a set of monoclonal antibodies that react specifically with the denatured forms of human laminin. Degradation of the ECM during angiogenesis has been proposed to expose cryptic epitopes (“cryptic” epitopes) in the ECM that are only exposed upon proteolytic remodeling of the ECM. Further, it has been suggested that some exposed cryptic epitopes of the ECM play a critical and essential role in angiogenesis by engaging cell surface change receptors on endothelial cells and that the subsequent signaling provides an essential change in endothelial cell behavior. The central hypothesis of this proposal is that cryptic sites in laminin are essential for angiogenesis. A corollary of this hypothesis is that antagonists of such cryptic sites will alter endothelial cell behavior and block angiogenesis.

I. Laminin

We have generated a panel of monoclonal antibodies against laminin and screened for antibodies that preferentially bind to denatured laminin.

RESULTS: Three mice were chosen for the immunization with laminin. Preimmune serum was first collected as a reference point for later ELISA analysis. Mice were immunized with “native” (not thermally denatured) laminin. Two days later mice were treated with cyclophosphamide to kill proliferating B-cells that were stimulated by native laminin. This was repeated three times to eliminate B-cells that respond to native laminin. Mice were then immunized with thermally denatured laminin to evoke an immune response for denatured laminin. After several cycles of immunization with denatured laminin, serum was collected for ELISA analysis. The serum from two of the three mice preferentially reacted with heat denatured laminin rather than native laminin (Table 1).

TABLE 1
Solid phase ELISA analysis of mouse serum against
native and thermally denatured human laminin.

OD (492 nm)

| | Native laminin | Denatured laminin |
|---|---|---|
| Mouse 1 | 0.15 | 0.18 |
| Mouse 2 | 0.18 | 0.49 |
| Mouse 3 | 0.27 | 0.56 |

Mouse #2 and #3 showed preferential reactivity for thermally denatured laminin and were therefore chosen for generation of hybridomas. Spleens were collected and fused with an immortalized fusion partner to generate a pool of hybridomas. The pool was seeded as single cells to generate clonal populations. The cloning was repeated a second time to assure clonality. Culture supernatants from each clone was tested for reactivity for native and thermally denatured laminin. The vast majority of clones, >850 of 1022 clones, showed less that a 1.2-fold preference for denatured laminin. Approximately 50 clones were identified whose culture supernatants preferentially reacted (>1.6-fold preference) with denatured laminin. These clones were expanded and the isotype of the antibody produced was determined by standard ELISA analysis. All clones were found to be IgM. Culture supernatants of these 50 clones were prepared and IgM antibody purified by affinity to an IgM-binding lectin. The protein concentration of the IgM preparations was determined by Bradford analysis.

ELISA analysis of the 50 samples of purified IgM showed that 10 showed a strong preference for denatured laminin and another 10 showed a modest preference for denatured laminin (Table 2)

TABLE 2
Purified IgM (5 ug/ml) was analyzed by ELISA for reactivity
to native and denatured laminin. ELISA plates were coated
with native or denatured laminin (10 ug/ml) over night at
4 C. A standard ELISA protocol was used. Triplicate samples
were analyzed and the average OD determined. For a negative
control, no primary IgM was used in the ELISA.

OD 492

| Clone # | native | denatured | fold preference |
|---|---|---|---|
| LMD1 | 0.061 | 0.118 | 1.9 |
| LMD2 | 0.019 | 0.116 | 6 |
| LMD5 | 0.127 | 0.236 | 1.9 |
| LMD9 | 0.097 | 0.201 | 2 |
| LMD11 | 0.114 | 0.219 | 1.9 |
| LMD13 | 0.124 | 0.229 | 1.8 |
| LMD14 | 0.105 | 0.173 | 1.6 |
| LMD15 | 0.116 | 0.209 | 1.8 |
| LMD16 | 0.104 | 0.181 | 1.8 |
| LMD17 | 0.131 | 0.243 | 1.9 |
| LMD21 | 0.103 | 0.228 | 2.2 |
| LMD23 | 0.130 | 0.229 | 1.8 |
| LMD24 | 0.104 | 0.205 | 2.0 |
| LMD26 | 0.150 | 0.278 | 1.9 |
| LMD30 | 0.181 | 0.286 | 1.6 |
| LMD52 | 0.074 | 0.198 | 2.7 |
| LMD105 | 0.107 | 0.228 | 2.1 |
| LMD209 | 0.173 | 0.302 | 1.7 |
| LMD418 | 0.291 | 0.381 | 1.7 |

### Example 3

### Further Characterization

Ascites fluid is produced for each of the hybridomas shown in table 2. Antibody are purified from each of the acites by affinity to a IgM-binding column. The purified antibodies are analyzed for the ability to alter endothelial cell behavior on native and denatured laminin. Specifically, antibodies are assayed for the ability to slow the rate of endothelial cell adhesion to and migration on denatured laminin while that on native laminin is not dramatically affected. Among such antibodies will be antibodies that block endothelial cell adhesion and/or migration in vivo and block angiogenesis and tumor growth.

### Example 4

### Obtaining Antibodies to Other Proteins

I. Fibronectin

Three mice were chosen for the immunization with fibronectin. Preimmune serum was first collected as a reference point for later ELISA analysis. Mice were immunized with “native” (not thermally denatured) fibronectin. Two days later mice were treated with cyclophosphamide to kill proliferating B-cells that were stimulated by native fibronectin. Mice were then immunized with thermally denatured fibronectin to evoke an immune response for denatured fibronectin. Mice were then immunized with thermally denatured fibronectin to evoke an immune response for denatured fibronectin. After several cycles of immunization with denatured fibronectin, serum was collected for ELISA analysis. The serum from the three mice preferentially reacted with heat-denatured fibronectin rather than native fibronectin (Table 3).

TABLE 3
Solid phase ELISA analysis of mouse serum against
native and thermally denatured human fibronectin.

OD (492 nm)

| | Native fibronectin | Denatured fibronectin |
|---|---|---|
| Mouse 1 | 0.33 | 0.56 |
| Mouse 2 | 0.14 | 0.39 |
| Mouse 3 | 0.11 | 0.30 |

Each of the three mice were sacrificed and their spleens used to production of a pool of hybridomas. The pool of hybridomas was frozen for analysis at a later date.

### Example 5

### Cell Adherence/Migration Assays

Identification of monoclonal antibodies which inhibit vascular endothelial cell adhesion and migration on native or denatured Type V collagen laminin and fibronectin. For each of the antigens (Type V collagen, laminin, and fibronectin), monoclonal antibodies are found that preferentially recognize the denatured antigen. These monoclonal antibodies are tested for their ability to inhibit adhesion and migration of HUVECs in the denatured ECM proteins.

Cell Adhesion Assays. Native and thermally denatured Type V collagen, laminin, and fibronectin (25 μg/ml) are immobilized on 96-well non-tissue culture treated plates by incubation at 4° C. for 16 hr (Damsky, C. H., and Werb, Z. (1992) Current Opinion in Cell Biology 4(5), 772-81). Wells are then washed and incubated with 1% BSA in PBS for 1 hour at 37° C. Subconfluent HUVECs are harvested, washed and resuspended in adhesion buffer (RPMI 1640, 1 mM MgCl₂, 0.2 mM MnCl₂, and 0.5% BSA). HUVECs (1×10⁵ cells) resuspended in 200 μl of the adhesion buffer are added to each well and allowed to attach for 30 minutes at 37° C. Monoclonal antibodies to be tested (and control isotype matched monoclonal antibodies) are added to the adhesion buffer at a final concentration of 25 μg/ml. The non-attached cells are removed by gentle washing and the attached cells are stained for 10 minutes with crystal violet as described (Damsky, C. H., and Werb, Z. (1992) Current Opinion in Cell Biology 4(5), 772-81). The wells are washed three times with PBS and cell associated crystal violet is eluted by addition of 100 of 10% acetic acid. Assays are performed in triplicates. Cell adhesion is quantified by measuring the optical density of eluted crystal violet at a wavelength of 600 nm with microtiter plate reader.

Cell Migration Assays. The under side of Transwell Membranes (8.0 μm pore size) is coated with native, or thermally denatured Type V collagen, laminin, or fibronectin at a concentration of 25 μg/ml in PBS for 16 hours at 4° C. Six hundred microliters (600 μl) of migration buffer (RPMI 1640, 1 mM MgCl₂, 0.2 mM MnCl₂, and 0.5% BSA) is added to the lower chamber. HUVECs (1×10⁵ cells) are resuspended in 100 μl of migration buffer, placed in the upper chamber, and allowed to migrate for 8 hr at 37° C. Cells remaining on the upper side of the membrane are removed with a cotton swab. Cells migrated to the underside are fixed and stained with crystal violet. The Transwell membranes are washed and the cell associated crystal violet is eluted with 10% acetic acid as described (Damsky & Werb, 1992). Cell migration is quantified by measuring the optical density of eluted crystal violet at a wavelength of 600 nm with microtiter plate reader.

Anticipated results, limitations and alternatives: Some of the monoclonal antibodies identified will inhibit adhesion and/or migration of HUVECs to the antigens. However, denaturation of the ECM proteins may cause reduction in cell adhesion, in which case assay conditions may be adjusted by increasing the amount of the denatured protein immobilized on the plate to optimize cell binding and migration. The amount of protein which supports >70% of cell binding will be employed for plate coating.

### Example 6

### Inhibition of Angiogenesis/Angiogenesis Assays

Antagonists of the invention also can be assayed for their ability to modulate angiogenesis in a tissue. Any suitable assay known to one of skill in the art can be used to monitor such effects. Several such techniques are described herein.

One assay that measures angiogenesis in the chick chorioallantoic membrane (CAM) is known as the CAM assay. The CAM assay has been described in detail by others, and further has been used to measure both angiogenesis and neovascularization of tumor tissues. See Ausprunk et al., Am. J. Pathol., 79:597-618 (1975) and Ossonski et al., Cancer Res., 40:2300-2309 (1980).

The CAM assay is a well recognized assay model for in vivo angiogenesis because neovascularization of whole tissue is occurring, and actual chick embryo blood vessels are growing into the CAM or into the tissue grown on the CAM. The CAM assay illustrates inhibition of neovascularization based on both the amount and extent of new vessel growth.

Furthermore, it is easy to monitor the growth of any tissue transplanted upon the CAM, such as a tumor tissue. Finally, the assay is particularly useful because there is an internal control for toxicity in the assay system. The chick embryo is exposed to any test reagent, and therefore the health of the embryo is an indication of toxicity.

Another assay that measures angiogenesis is the in vivo rabbit eye model and is referred to as the rabbit eye assay. The rabbit eye assay has been described in detail by others, and further has been used to measure both angiogenesis and neovascularization in the presence of angiogenic inhibitors such as thalidomide. See D'Amato et al. (1994) Proc. Natl. Acad. Sci. 91:4082-4085.

The rabbit eye assay is a well recognized assay model for in vivo angiogenesis because the neovascularization process, exemplified by rabbit blood vessels growing from the rim of the cornea into the cornea, is easily visualized through the naturally transparent cornea of the eye. Additionally, both the extent and the amount of stimulation or inhibition of neovascularization or regression of neovascularization can easily be monitored over time.

Finally, the rabbit is exposed to any test reagent, and therefore the health of the rabbit is an indication of toxicity of the test reagent.

A further assay that measures angiogenesis in the chimeric mouse:human mouse model and is referred to as the chimeric mouse assay. The assay has been described in detail by others, and further has been described herein to measure angiogenesis, neovascularization, and regression of tumor tissues. See Yan, et al. (1993) J. Clin. Invest. 91:986-996.

The chimeric mouse assay is a useful assay model for in vivo angiogenesis because the transplanted skin grafts closely resemble normal human skin histologically and neovascularization of whole tissue is occurring wherein actual human blood vessels are growing from the grafted human skin into the human tissue on the surface of the grafted human skin. The origin of the neovascularization into the human graft can be demonstrated by immunohistochemical staining of the neovasculature with human-specific endothelial cell markers.

The chimeric mouse assay demonstrates regression of neovascularization based on both the amount and extent of regression of new vessel growth. Furthermore, it is easy to monitor effects on the growth of any tissue transplanted upon the grafted skin, such as a tumor tissue. Finally, the assay is useful because there is an internal control for toxicity in the assay system. The chimeric mouse is exposed to any test reagent, and therefore the health of the mouse is an indication of toxicity.

The invention illustratively described herein can suitably be practiced in the absence of any element or elements, limitation or limitations that is not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalent of the invention shown or portion thereof, but it is recognized that various modifications are possible within the scope of the invention claimed. Thus, it should be understood that although the present invention has been specifically disclosed by preferred embodiments and optional features, modifications and variations of the inventions embodied herein disclosed can be readily made by those skilled in the art, and that such modifications and variations are considered to be within the scope of the inventions disclosed herein. The inventions have been described broadly and generically herein. Each of the narrower species and subgeneric groupings falling within the generic disclosure also form the part of these inventions. This includes within the generic description of each of the inventions a proviso or negative limitation that will allow removing any subject matter from the genus, regardless or whether or not the material to be removed was specifically recited. In addition, where features or aspects of an invention are described in terms of the Markush group, those schooled in the art will recognize that the invention is also thereby described in terms of any individual member or subgroup of members of the Markush group. Further, when a reference to an aspect of the invention lists a range of individual members, as for example, ‘SEQ ID NO:1 to SEQ ID NO:100, inclusive,’ it is intended to be equivalent to listing every member of the list individually, and additionally it should be understood that every individual member may be excluded or included in the claim individually.

The steps depicted and/or used in methods herein may be performed in a different order than as depicted and/or stated. The steps are merely exemplary of the order these steps may occur. The steps may occur in any order that is desired such that it still performs the goals of the claimed invention.

From the description of the invention herein, it is manifest that various equivalents can be used to implement the concepts of the present invention without departing from its scope. Moreover, while the invention has been described with specific reference to certain embodiments, a person of ordinary skill in the art would recognize that changes can be made in form and detail without departing from the spirit and the scope of the invention. The described embodiments are considered in all respects as illustrative and not restrictive. It should also be understood that the invention is not limited to the particular embodiments described herein, but is capable of many equivalents, rearrangements, modifications, and substitutions without departing from the scope of the invention. Thus, additional embodiments are within the scope of the invention and within the following claims.

All U.S. patents and applications; foreign patents and applications; scientific articles; books; and publications mentioned herein are hereby incorporated by reference in their entirety as if each individual patent or publication was specifically and individually indicated to be incorporated by reference, including any drawings, figures and tables, as though set forth in full.

1. A monoclonal antibody that has at least about a 1.5-fold increased binding to denatured laminin when compared to said monoclonal antibody's binding to native laminin.

2. The monoclonal antibody of claim 1 wherein said increased binding is at least about a two-fold difference in binding between denatured laminin compared to said monoclonal antibody's binding to native laminin.

3. The monoclonal antibody of claim 1 wherein said increased binding is at least about a six-fold difference in binding between denatured laminin compared to said monoclonal antibody's binding to native laminin.

4. A monoclonal antibody that specifically binds to a backbone domain in denatured laminin with an affinity that is greater by a factor of at least about 1.5 than said monoclonal antibody binds to said backbone domain in the native form of said laminin.

5. The monoclonal antibody of claim 4 wherein said affinity of binding to said backbone domain is at least about two-fold greater in denatured laminin when compared to native laminin.

6. The monoclonal antibody of claim 4 wherein said affinity of binding to said backbone domain is at least about six-fold greater in denatured laminin when compared to native laminin.

7. The monoclonal antibody of claim 1 or claim 2 wherein said monoclonal antibody is a monoclonal antibody having the binding specificity of monoclonal antibody clone LMD2, LMD9, LMD21, LMD24, LMD52, LMD105, LMD1, LMD5, LMD11, LMD17, LDM26, LMD13, LMD14, LMD15, LMD16, LMD23, LMD30, LMD209, or LMD418.

8. The monoclonal antibody of claim 1 or claim 2 wherein said monoclonal antibody is a monoclonal antibody having the binding specificity of monoclonal antibody clone LMD9.

9. The monoclonal antibody of claim 1 or claim 2 wherein said monoclonal antibody is a monoclonal antibody having the binding specificity of monoclonal antibody clone LMD11.

10. The monoclonal antibody of claim 1 or claim 2 wherein said monoclonal antibody is a monoclonal antibody having the binding specificity of monoclonal antibody clone LMD17.

11. The monoclonal antibody of claim claim 1 or claim 2 wherein said monoclonal antibody is a humanized monoclonal antibody.

12. The monoclonal antibody of claim 1 or claim 2 wherein said monoclonal antibody is a chemically modified monoclonal antibody.

13. The monoclonal antibody of claim 1 or claim 2 wherein said monoclonal antibody is conjugated to a cytotoxic agent or a cytostatic agent.

14. A method of inhibiting angiogenesis in a tissue comprising the step of administering to said tissue said monoclonal antibody of any one of claims 1 through 10.

15. The method of claim 14 wherein said monoclonal antibody is administered in conjunction with chemotherapy.

16. The method of claim 14 wherein said monoclonal antibody is administered in conjunction with radiation therapy.

17. The method of claim 14 wherein said monoclonal antibody is conjugated with a radioisotope.

18. The method of claim 14 wherein said tissue is inflamed and angiogenesis is occurring.

19. The method of claim 14 wherein said tissue is arthritic tissue, ocular tissue, retinal tissue, or a hemangioma.

20. A method of inhibiting tumor growth, tumor metastasis, or metastasized tumor growth in a tissue comprising administering the monoclonal antibody of any one of claims 1 through 11.

21. The method of claim 20 wherein said monoclonal antibody is administered in conjunction with chemotherapy.

22. The method of claim 20 wherein said monoclonal antibody is administered in conjunction with radiation therapy.

23. The method of claim 20 wherein said monoclonal antibody is conjugated to a radioisotope prior to being administered.

24. The method of claim 20 wherein said treatment comprises the step of heating the tissue.

25. A method of inhibiting psoriasis, macular degeneration, restenosis, rheumatoid arthritis, scleraderma, fibrosis, or vasculitis in a tissue comprising administering said monoclonal antibody of any one of claims 1 through 11.

26. The method of claim 25 wherein said monoclonal antibody is administered in conjunction with chemotherapy.

27. The method of claim 25 wherein said treatment also comprises the step of heating the tissue.

28. A purified peptide consisting essentially of an epitope identified by the binding of a monoclonal antibody of the invention.

29. The purified peptide of claim 28 wherein said monoclonal antibody is a monoclonal antibody having the binding specificity of monoclonal antibody clone LMD2, LMD9, LMD21, LMD24, LMD52, LMD105, LMD1, LMD5, LMD11, LMD17, LDM26, LMD13, LMD14, LMD15, LMD16, LMD23, LMD30, LMD209, or LMD418.

30. The purified peptide of claim 28 wherein said monoclonal antibody is a monoclonal antibody having the binding specificity of monoclonal antibody clone LMD9.

31. The purified peptide of claim 28 wherein said monoclonal antibody is a monoclonal antibody having the binding specificity of monoclonal antibody clone LMD11.

32. The purified peptide of claim 28 wherein said monoclonal antibody is a monoclonal antibody having the binding specificity of monoclonal antibody clone LMD17.
